(19) United States
(12) Patent Application Publication
(10) Pub. No.: US 2014/0073580 A1
(43) Pub. Date: Mar. 13, 2014

(54) COMBINATIONS OF HDAC INHIBITORS AND PROTEASOME INHIBITORS

(71) Applicants: Lia GORE, Denver, CO (US); Deborah DERYCKERE, Boulder, CO (US)

(72) Inventors: Lia GORE, Denver, CO (US); Deborah DERYCKERE, Boulder, CO (US)

(21) Appl. No.: 13/940,110

(22) Filed: Jul. 11, 2013

Related U.S. Application Data
(63) Continuation of application No. 12/273,350, filed on Nov. 18, 2008, now abandoned.
(60) Provisional application No. 60/989,063, filed on Nov. 19, 2007.

(51) Int. Cl. A61K 38/07; A61K 31/44; A61K 31/506; A61K 31/69

(52) U.S. Cl. CPC A61K 38/07; A61K 31/69; A61K 31/44; A61K 31/506
USPC 514/19.3; 514/64

(57) ABSTRACT

Provided herein are pharmaceutical agents, pharmaceutical compositions, methods of treatment, treatment regimens and kits for the treatment of cancer.

### CROSS-REFERENCE

This application is a continuation of application Ser. No. 12/273,350, filed Nov. 18, 2008, which claims priority to provisional Application No. 60/989,063, filed on Nov. 19, 2007, each of which is incorporated by reference in its entirety.

### BACKGROUND OF THE INVENTION

Cancer is the leading cause of death worldwide. In 2005, cancer accounted for 7.6 million (or 13% of all) deaths.

### SUMMARY OF THE INVENTION

Accordingly, provided herein are combinations, pharmaceutical compositions, kits, treatment regimens and methods of treating diseases. Certain embodiments of the present invention provide a method for treating cancer comprising administering to a patient a therapeutically effective amount of a Class I selective HDAC inhibitor and a proteasome inhibitor.

In some embodiments, the proteasome inhibitor is selected from, by way of non-limiting example, bortezomib (Velcade, PS-341), PR-171 (carfilzomib), (benzyloxycarbonyl)-Leu-Leu-phenylalaninal, 2,3,5a,6-tetrahydro-6-hydroxy-3-(hydroxymthyl)-2-methyl-10H-3a,10a-epidithio-pyrazinol[1,2α]indole-1,4-dione, 4-hydroxy-3-nitrophenylacetyl-Leu-Leu-Leu-vinyl sulphone, sapojargon, Ac-hFLFL-epoxide, aclacinomycin A, aclarubicin, ACM, AdaK(Bio)Ahx₃L₃VS, AdaLys(Bio)Ahx₃L₃VS, Adamantane-acetyl-(6-aminohexanoyl)-3-(leucunyl)-3-vinyl-(methyl)-sulphone, ALLM, ALLN, Calpain Inhibitor I, Calpain Inhibitor II, Carbobenzoxy-L-leucyl-L-leucyl-L-leucinal, Carbobenzoxy-L-leucyl-L-leucyl-L-norvalinal, gliotoxin, isovalery-L-tyrosyl-L-valyl-DL-tyrosinal, clasto-lactacystin-β-lactone, Z-LL-Nva-CHO, Ubiquitin Aldehyde, YU101, MP-LLL-VS, LDN-57444, Z-GPFL-CHO, Z-LLL-CHO, lovastatin, α-methyl-clasto-lactacystin-β-lactone, mevinolin, MK-803, NIP-L₃VS, NP-LLL-VS, NPI-0052 (salinosporamide A), MLN519 (PS-519), NLVS (trileucine vinyl-sulfone), ritonavir, Ro106-9920, Z-LLF-CHO, Z-LL-B(OH)₂, RRRPRPPYLPR, Tyropeptin A, ZL₃VS, PR-11, PR-39, 0106-9920, Proteasome Inhibitor I, Proteasome Inhibitor II, Proteasome Inhibitor III, Proteasome Inhibitor IV, AdaAhx3L3VS, efrapeptin, MG-132, MG-262, MG-115, α-methylomuralide, MG-101, epoxomicin, omuralide, lactacystin andor NEOSH101. In some embodiments, the proteasome inhibitor is selected from, by way of non-limiting example, bortezomib (Velcade, PS-341), PR-171 (carfilzomib), and NPI-0052 (salinosporamide A). In a specific embodiment, the proteasome inhibitor is bortezomib.

In some embodiments, the Class I selective HDAC inhibitor is selected from, by way of non-limiting example, N-(2-amino-phenyl)-4-[(4-pyridin-3-yl-pyrimidin-2-ylamino)-methyl]-benzamide (MGCD-0103), N-(2-aminophenyl)-4-(N-(pyridin-3-ylmethoxycarbonyl)aminomethyl)benzamide (MS-275, SNDX-275), FK228, spiruchostatin A, SK7041, SK7068 and 6-amino nicotinamides. In some embodiments, the Class I selective HDAC is selected from N-(2-amino-phenyl)-4-[(4-pyridin-3-yl-pyrimidin-2-ylamino)-methyl]-benzamide (MGCD-0103), N-(2-aminophenyl)-4-(N-(pyridin-3-ylmethoxycarbonyl)aminomethyl)benzamide (MS-275, SNDX-275), FK228, spiruchostatin A, SK7041, SK7068 and 6-amino nicotinamides. In a specific embodiment, the Class I selective HDAC inhibitor is N-(2-aminophenyl)-4-(N-(pyridin-3-ylmethoxycarbonyl)aminomethyl)benzamide. In another specific embodiment, the Class I selective HDAC inhibitor is N-(2-amino-phenyl)-4-[(4-pyridin-3-yl-pyrimidin-2-ylamino)-methyl]-benzamide.

In certain embodiments, the Class I selective HDAC inhibitor forces G₁ arrest.

In some embodiments of the present invention, the proteasome inhibitor is administered after the Class I selective HDAC inhibitor.

In various embodiments of the present invention, the cancer treated by the method disclosed herein is, by way of non-limiting example, cancer is brain cancer, breast cancer, lung cancer, ovarian cancer, pancreatic cancer, prostate cancer, renal cancer, colorectal cancer, leukemia, myeloid leukemia, acute myeloid leukemia (AML), glioblastoma, follicular lymphoma, pre-B acute leukemia, chronic lymphocytic B-leukemia, mesothelioma, small cell lung cancer, multiple myeloma, leukemia, myelodysplastic syndromes, lymphomas, acute leukemia, acute lymphocytic leukemia (ALL), acute myeloid leukemia (AML)/acute nonlymphocytic leukemia (ANLL), chronic lymphocytic leukemia (CLL), chronic myelogenous leukemia (CML), Hodgkin's lymphoma, non-Hodgkin's lymphoma, cutaneous t-cell lymphoma (CTCL), pediatric acute leukemia, pediatric acute myeloid leukemia, pediatric acute lymphoid leukemia, juvenile myelomonocytic leukemia (JJMML/JCML), mantle cell lymphoma (MCL), cancers of the head and neck, cancers of the reproductive system, cancers of the gastro-intestinal system, cancers of the urinary system, cancer of the upper digestive tract or colorectal cancer, bladder cancer, renal cell carcinoma, ovarian cancer, lymphoid leukemia. In some embodiments, the cancer is selected from, by way of non-limiting example, multiple myeloma, non-small cell lung cancer, acute myeloid leukemia, lymphoblastic lymphoma, follicular lymphoma, non-Hodgkin's lymphoma, mantle cell lymphoma, lung cancer, Hodgkin's lyphoma, head and neck cancer, colorectal cancer, ovarian cancer, leukemia, prostrate cancer, melanoma, bladder cancer, kidney cancer, lung cancer, sarcoma, gastric cancer, pancreatic cancer, liver cancer, gastrointestinal cancer, cervical cancer and breast cancer.

In some embodiments, the method for treating cancer by administering to a patient a therapeutically effective amount of a Class I selective HDAC inhibitor and a proteasome inhibitor further includes the step of administering at least one additional cancer therapy to the patient. In certain embodiments, the additional cancer therapy is selected from surgery or radiation therapy. In some embodiments, the additional cancer therapy is administration of a second chemotherapeutic agent. In specific embodiments, the chemotherapeutic agent is adriamycin, gemcitabine, mitomycin C, cisplatin, carboplatin, oxaliplatin, fluorouracil, leucovorin, cytarabine, etoposide, capecitabine, temozolomide, doxorubicin, daunomycin, daunorubicin, paclitaxel, docetaxel, cyclophosphamide, ifosfamide, methotrexate, bevacizumab or trastuzumab.

In certain embodiments of the present invention, the HDAC inhibitor sensitizes the cancer cells to the proteasome inhibitor.

In various embodiments, the present invention provides for a method for treating cancer by administering to a patient a therapeutically effective amount of N-(2-aminophenyl)-4-(N-(pyridin-3-ylmethoxycarbonyl)aminomethyl)benzamide and bortezomib. In specific embodiments, the N-(2-aminophenyl)-4-(N-(pyridin-3-ylmethoxycarbonyl)aminomethyl)benzamide is administered after the bortezamide.

In other embodiments, the present invention provides for a method for treating cancer by administering to a patient a therapeutically effective amount of N-(2-aminophenyl)-4-(N-(pyridin-3-ylmethoxycarbonyl)aminomethyl)benzamide and salinosporamide A. In a specific embodiment, the N-(2-aminophenyl)-4-(N-(pyridin-3-ylmethoxycarbonyl)aminomethyl)benzamide is administered after the salinosporamide A.

In still other embodiments, the present invention provides for a kit containing a therapeutically effective amount of a Class I selective HDAC inhibitor and a proteasome inhibitor. In various embodiments, the kit contains a proteasome inhibitor selected from, by way of non-limiting example, bortezomib (Velcade, PS-341), PR-171 (carfilzomib), (benzyloxycarbonyl)-Leu-Leu-phenylalaninal, 2,3,5a,6-tetrahydro-6-hydroxy-3-(hydroxymthyl)-2-methyl-10H-3a,10a-epidithio-pyrazinol[1,2α]indole-1,4-dione, 4-hydroxy-3-nitrophenylacetyl-Leu-Leu-Leu-vinyl sulphone, sapojargon, Ac-hFLFL-epoxide, aclacinomycin A, aclarubicin, ACM, AdaK(Bio)Ahx₃L₃VS, AdaLys(Bio)Ahx₃L₃VS, Adamantane-acetyl-(6-aminohexanoyl)-3-(leucunyl)-3-vinyl-(methyl)-sulphone, ALLM, ALLN, Calpain Inhibitor I, Calpain Inhibitor II, Carbobenzoxy-L-leucyl-L-leucyl-L-leucinal, Carbobenzoxy-L-leucyl-L-leucyl-L-norvalinal, gliotoxin, isovalery-L-tyrosyl-L-valyl-DL-tyrosinal, clasto-lactacystin-β-lactone, Z-LL-Nva-CHO, Ubiquitin Aldehyde, YU101, MP-LLL-VS, LDN-57444, Z-GPFL-CHO, Z-LLL-CHO, lovastatin, α-methyl-clasto-lactacystin-β-lactone, mevinolin, MK-803, NIP-L3VS, NP-LLL-VS, NPI-0052 (salinosporamide A), MLN519 (PS-519), NLVS (trileucine vinyl-sulfone), ritonavir, Ro106-9920, Z-LLF-CHO, Z-LL-B(OH)₂, RRRPRPPYLPR, Tyropeptin A, ZL₃VS, PR-11, PR-39, 0106-9920, Proteasome Inhibitor I, Proteasome Inhibitor II, Proteasome Inhibitor III, Proteasome Inhibitor IV, AdaAhx3L3VS, efrapeptin, MG-132, MG-262, MG-115, α-methylomuralide, MG-101, epoxomicin, omuralide, lactacystin andor NEOSH101. In various embodiments, the kit contains a Class I selective HDAC inhibitor is selected from, by way of non-limiting example, N-(2-amino-phenyl)-4-[(4-pyridin-3-yl-pyrimidin-2-ylamino)-methyl]-benzamide (MGCD-0103), N-(2-aminophenyl)-4-(N-(pyridin-3-ylmethoxycarbonyl)aminomethyl)benzamide (MS-275, SNDX-275), FK228, spiruchostatin A, SK7041, SK7068 and 6-amino nicotinamides.

In specific embodiments, the dosage form of the selective HDAC inhibitor and the dosage form of the proteasome inhibitor are different colors. In other embodiments, the kit contains at least one dosage form with the Class I selective HDAC inhibitor and the proteasome inhibitor.

### BRIEF DESCRIPTION OF THE DRAWINGS

FIG. 1 illustrates the dose response of various cancer cell lines for SNDX-275 (entinostat) and SAHA (vorinostat).

FIG. 2A illustrates the mean IC₅₀ values of SAHA and SNDX-275 against various cancer cell lines. FIG. 2B illustrates the mean IC₅₀ values of SAHA, SNDX-275, and bortezomib against various cancer cell lines.

FIG. 3 illustrates the dose dependency of SNDX-275 (entinostat) and SAHA (vorinostat) mediated cell death for various cancer cell lines.

FIG. 4A illustrates the percentages of dead cancer cells found in samples that are untreated, treated with an HDAC inhibitor, treated with a proteasome inhibitor, or treated with an HDAC inhibitor and a proteasome inhibitor. FIG. 4B illustrates the percentages of viable cancer cells found in samples that are untreated, treated with an HDAC inhibitor, treated with a proteasome inhibitor, or treated with an HDAC inhibitor and a proteasome inhibitor.

FIGS. 5A and 5B illustrate the synergistic effects in causing cancer cell death when treating such cancer cells with an HDAC inhibitor and a proteasome inhibitor.

### DESCRIPTION OF THE INVENTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention. It is intended that the claims define the scope of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Certain Terminology

Unless defined otherwise, all technical and scientific terms used herein have the same meaning as is commonly understood by one of skill in the art to which the claimed subject matter belongs. In the event that there is a plurality of definitions for terms herein, those in this section prevail.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

The HDACs are a family including at least eighteen enzymes, grouped in three classes (Class I, II and III). Class I HDACs include, but are not limited to, HADCs 1, 2, 3, 8 and 11. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not substantially interact with all three HDAC classes. As used herein, the term “Class I selective HDAC” refers to an HDAC inhibitor that does not substantially interact with a Class II HDAC or Class III HDAC.

The term “subject”, “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

Where combination treatments are contemplated, it is not intended that the agents described herein be limited by the particular nature of the combination. For example, the agents described herein may be administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. These also apply to cocktail therapies, e.g. the administration of three or more active ingredients.

As used herein, the terms “co-administration”, “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount”, “therapeutically effective amount” or “pharmaceutically effective amount” as used herein, refer to a sufficient amount of at least one agent being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising an agent as set forth herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the agents and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the agents described herein, and is relatively nontoxic, i.e., the material may be administered to an individual without causing undesirable biological effects or interacting in a deleterious manner with any of the components of the composition in which it is contained.

The term “carrier” as used herein, refers to relatively nontoxic chemical agents that facilitate the incorporation of an agent into cells or tissues.

The term “pharmaceutically acceptable derivative or prodrug” as used herein, refers to any pharmaceutically acceptable salt, ester, salt of an ester or other derivative of an agent, which, upon administration to a recipient, is capable of providing, either directly or indirectly, a agent of this invention or a pharmaceutically active metabolite or residue thereof. Particularly favored derivatives or prodrugs are those that increase the bioavailability of the agents of this invention when such agents are administered to a patient (e.g., by allowing an orally administered agent to be more readily absorbed into blood) or which enhance delivery of the parent agent to a biological compartment (e.g., the brain or lymphatic system).

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

As used herein, the terms “cancer treatment” “cancer therapy” and the like encompasses treatments such as surgery, radiation therapy, administration of chemotherapeutic agents and combinations of any two or all of these methods. Combination treatments may occur sequentially or concurrently. Treatments(s), such as radiation therapy and/or chemotherapy, that is administered prior to surgery, is referred to as neoadjuvant therapy. Treatments(s), such as radiation therapy and/or chemotherapy, administered after surgery is referred to herein as adjuvant therapy.

### Methods of Treatment

Histone deacetylation is a characteristic feature of cancer cells. Histones are small proteins that are tightly complexed with DNA to form a nucleosome, which is further connected by linker DNA to form a solenoid. Histones extending from the nucleosomal core are enzymatically modified, affecting chromatin structure and gene expression. Specifically, histones are modified by histone deacetylases (HDACs) by removing an acetyl group. The inhibition of HDACs is associated with cell cycle arrest (as well as increased differentiation and apoptosis and the inhibition of proliferation, angiogenesis and metastasis). In order to survive, a cell must pass through the cell cycle, which has four distinct phases: G1, S, G2 and M.

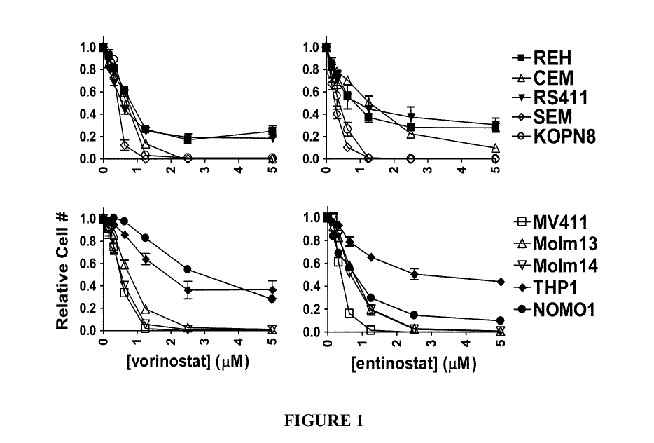

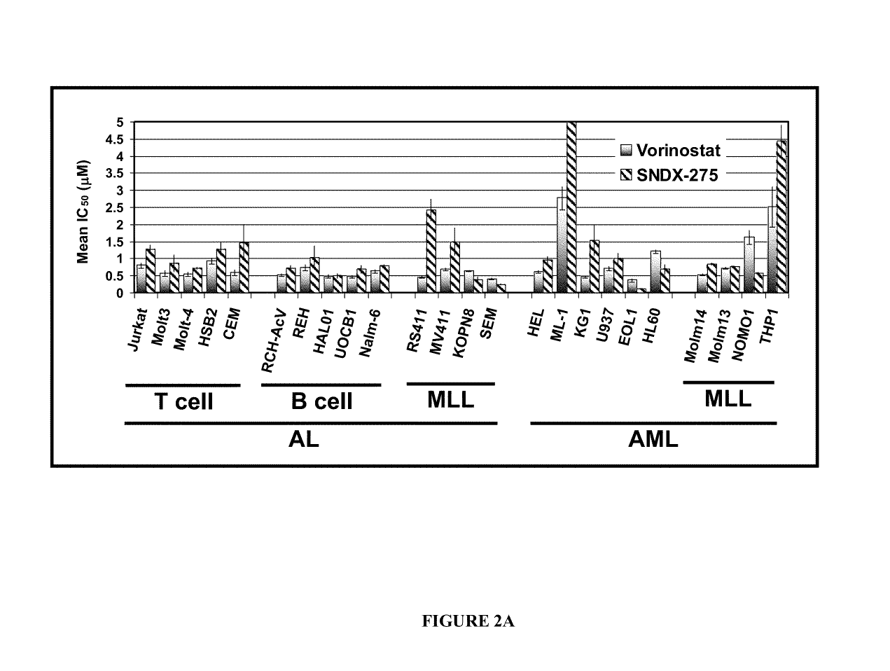

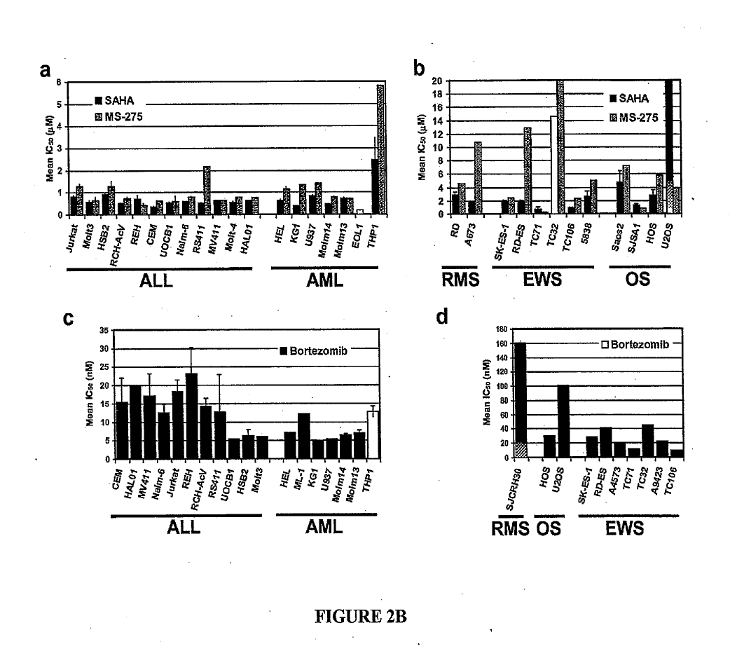

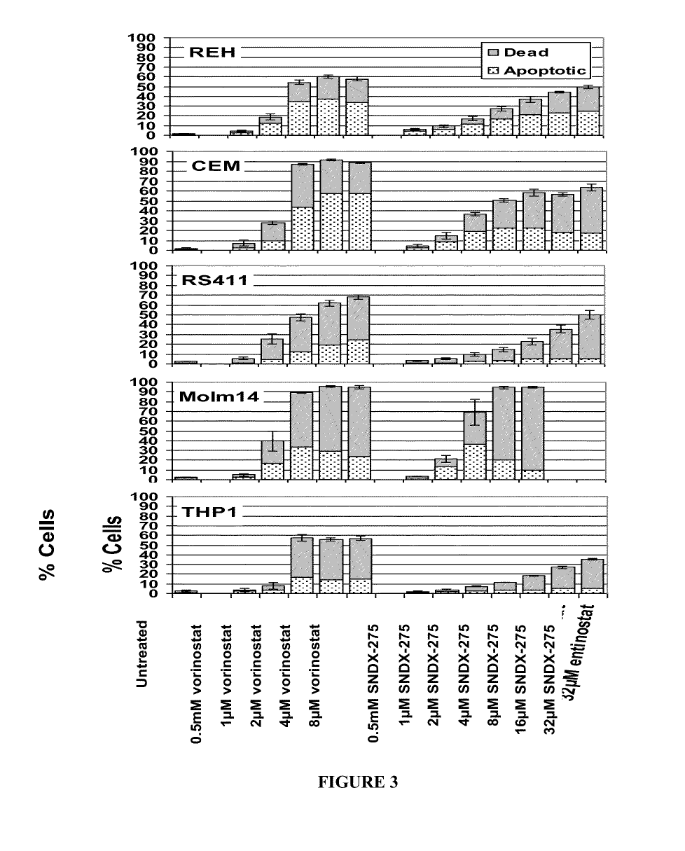

FIG. 1 illustrates the efficacy of HDAC inhibitors in treating various cancer cell lines at micromolar concentrations. FIGS. 2A and 2B further illustrates the efficacy of HDAC inhibitors and proteasome inhibitors in treating various cancer cell lines and includes mean IC₅₀ figures and comparisons for the HDAC inhibitors SNDX-275 and SAHA (N′-hydroxy-N-phenyl-octanediamide, suberoylanilide hydroxamic acid, or vorinostat). FIG. 3 illustrates the percentage of cancerous cell deaths caused by various concentrations of HDAC inhibitors SNDX-275 and SAHA.

In certain embodiments, the present invention provides for compositions and methods of treating a patient suffering from cancer by administering a therapeutically effective amount of a first agent and a second agent, wherein the first agent is an HDAC inhibitor.

In one embodiment provided herein is a method wherein the first agent is a Class I selective HDAC inhibitor. In certain embodiments, the HDAC inhibitor inhibits at least one of HDAC-1, HDAC-2, HDAC-3, HDAC-8, or HDAC-11. In a specific embodiment, the first agent inhibits HDAC-1. In another embodiment, the first agent inhibits HDAC-2. In yet another embodiment, the first agent inhibits HDAC-3. In still another embodiment, the first agent inhibits HDAC-11. In other embodiments, the first agent inhibits HDAC-1, HDAC-2, HDAC-3 and HDAC-11. In specific embodiments of the present invention the Class I selective HDAC inhibitor is, by way of non-limiting example, MGCD-0103 (N-(2-amino-phenyl)-4-[(4-pyridin-3-yl-pyrimidin-2-ylamino)-methyl]-benzamide), MS-275 (N-(2-aminophenyl)-4-(N-(pyridin-3-ylmethoxycarbonyl)aminomethyl)benzamide, SNDX-275), FK228, spiruchostatin A, SK7041, SK7068 and 6-amino nicotinamides.

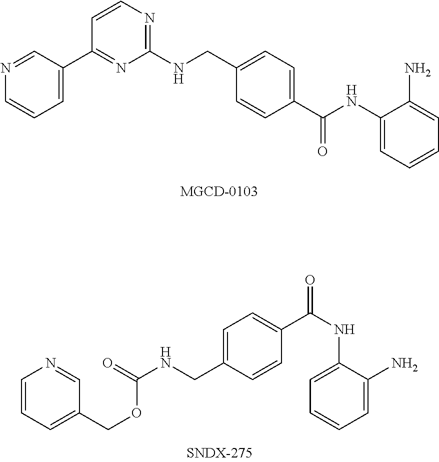 [SMILES: Nc1ccccc1NC(=O)c1ccc(CNc2nccc(-c3cccnc3)n2)cc1 ; Nc1ccccc1NC(=O)c1ccc(CNC(=O)OCc2cccnc2)cc1]

In another embodiment provided herein is a method wherein the first agent is an HDAC inhibitor that forces the arrest of a first cell cycle phase. In some embodiments, the first cell cycle phase that is arrested by the first agent is G1. In certain specific embodiments, the HDAC inhibitor that forces G1 arrest is, by way of non-limiting example, SNDX-275. In another non-limiting example, the HDAC inhibitor that forces G1 arrest is, by way of non-limiting example, MGCD-0103.

In some embodiments, the first agent is an HDAC inhibitor that alters the expression of at least one hematopoietic differentiation marker. In certain embodiments, the HDAC inhibitor that alters the expression of at least one hematopoietic differentiation marker is a Class I selective HDAC inhibitor. In some embodiments, the HDAC inhibitor alters the expression of at least one hematopoietic differentiation marker on AML. In various embodiments, the HDAC inhibitor alters the expression of at least one hematopoietic differentiation marker on ALL. In specific embodiments, the HDAC inhibitor that alters the expression of at least one hematopoietic differentiation marker is SNDX-275.

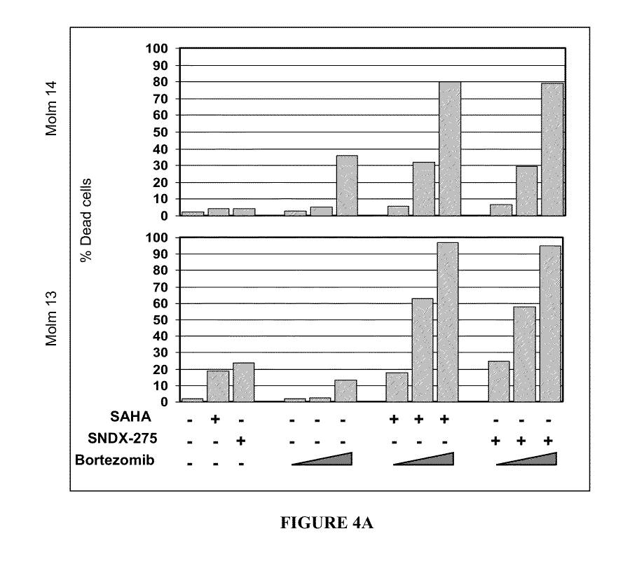

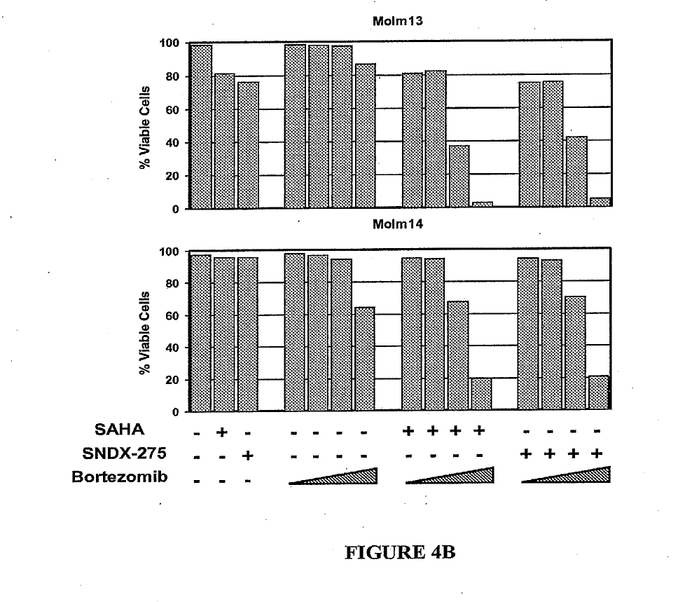

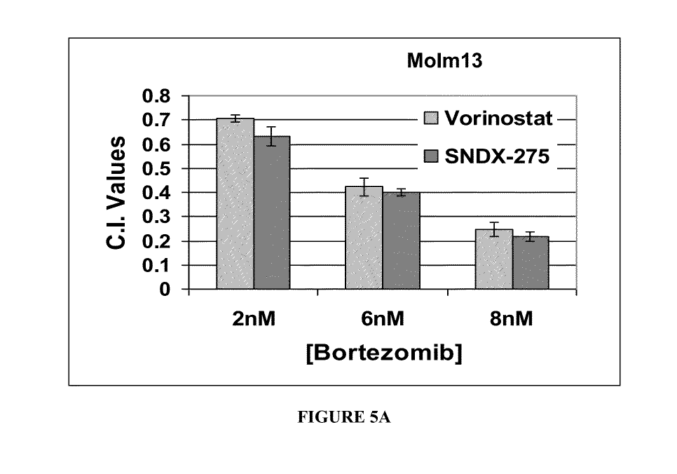

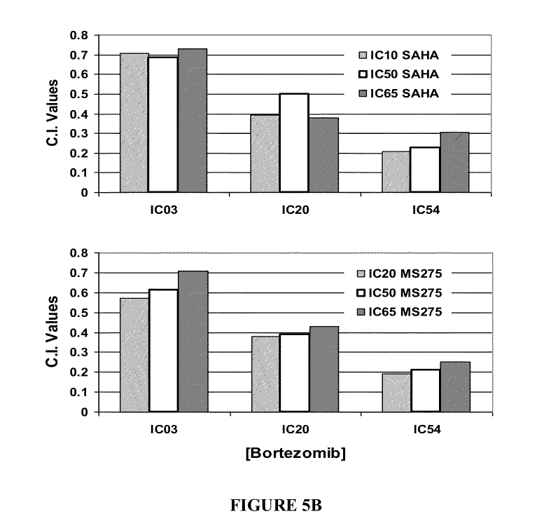

FIGS. 4A and 4B illustrate the effect on a cancer cell by combining an HDAC inhibitor with and without a proteasome inhibitor. According to the figures, the combination with either HDAC inhibitor SNDX-275 or SAHA with the proteasome inhibitor bortezomib greatly enhanced the ability of the treatment to cause cancerous cell death. In particular, FIGS. 4A and 4B illustrate the effect on leukemia cancers (e.g., cell lines Molm 14 and Molm 13). FIGS. 5A and 5B illustrate that the enhancement caused by the combination of the HDAC inhibitor with the proteasome inhibitor is synergistic (e.g., at inducing cancer cell death). This is accomplished by providing a combination index (C.I. value). In certain instances, the combination index (CI) is calculated by the following equation: CI=C_{A}/EC_{A}+C_{B}/EC_{B}, where C_{A} and C_{B} are the concentrations of the drugs A and B in the combination which elicits a certain effect and EC_{A} and EC_{B} are the isoeffective concentrations of the drugs A and B acting alone. If a combination index is less than 1, the combination is synergistic. If the combination index is equal to 1, the combination is additive. Finally, if a combination index is greater than 1, the combination is antagonistic. For example, in FIG. 5B, at IC₀₃ concentrations of bortezomib and IC₂₀ concentrations of SNDX-275, the C.I. value for the combination treatment is observed to be less than 0.6. At IC₅₄ concentrations of bortezomib and IC₂₀ concentrations of SNDX-275, the C.I. value for the combination treatment is observed to be less than 0.2. Accordingly, the combinations of selective HDAC inhibitors with proteasome inhibitors are highly synergistic in treating cancer.

In some embodiments, provided herein is an HDAC inhibitor and another anticancer agent (e.g., a proteasome inhibitor), wherein the effect of the combination on a cancer (e.g., any particular cancer, such as leukemia) is synergistic (e.g., has a C.I. value of less than 1). In certain embodiments, the synergistic effect is a synergistic ability to induce (including, e.g., resulting in or causing) cancer cell death. In specific embodiments, the C.I. value is less than 0.9, less than 0.8, less than 0.7, less than 0.6, less than 0.5, less than 0.4, less than 0.3, or less than 0.2. In certain embodiments, the effect of the combination is synergistic at about an IC₅₀ concentration of one or each agent (i.e., the IC₅₀ value when administered alone and/or in combination), or less. In some embodiments, the effect of the combination is synergistic at about an IC₄₀ concentration of one or each agent (i.e., the IC₄₀ value when administered alone and/or in combination), or less. In certain embodiments, the effect of the combination is synergistic at about an IC₃₀ concentration of one or each agent (i.e., the IC₃₀ value when administered alone and/or in combination), or less. In certain embodiments, the effect of the combination is synergistic at about an IC₂₀ concentration of one or each agent (i.e., the IC₂₀ value when administered alone and/or in combination), or less. In certain embodiments, the effect of the combination is synergistic at about an IC₆₅, or less; about an IC₅₀, or less; about an IC₄₅, or less; about an IC₂₀, or less; about an IC₁₀, or less; about an IC₀₅, or less; about an IC₀₄, or less; about an IC₀₃, or less; about an IC₀₂, or less; or about an IC₀₁, or less for one or each agent.

In some embodiments of the present invention the first agent is an HDAC inhibitor and the second agent is an anticancer agent that is not an HDAC inhibitor. In various embodiments, the first agent is a Class I selective HDAC inhibitor and/or an HDAC inhibitor that arrests G1. In some embodiments, the anticancer agent that is not an HDAC inhibitor is a proteasome inhibitor. In certain embodiments, the proteasome inhibitor is, by way of non-limiting example, bortezomib (Velcade, PS-341), PR-171 (carfilzomib), (benzyloxycarbonyl)-Leu-Leu-phenylalaninal, 2,3,5a,6-tetrahydro-6-hydroxy-3-(hydroxymthyl)-2-methyl-10H-3a,10a-epidithio-pyrazinol[1,2α]indole-1,4-dione, 4-hydroxy-3-nitrophenylacetyl-Leu-Leu-Leu-vinyl sulphone, sapojargon, Ac-hFLFL-epoxide, aclacinomycin A, aclarubicin, ACM, AdaK(Bio)Ahx₃L₃VS, AdaLys(Bio)Ahx₃L₃VS, Adamantane-acetyl-(6-aminohexanoyl)-3-(leucunyl)-3-vinyl-(methyl)-sulphone, ALLM, ALLN, Calpain Inhibitor I, Calpain Inhibitor II, Carbobenzoxy-L-leucyl-L-leucyl-L-leucinal, Carbobenzoxy-L-leucyl-L-leucyl-L-norvalinal, gliotoxin, isovalery-L-tyrosyl-L-valyl-DL-tyrosinal, clasto-lactacystin-β-lactone, Z-LL-Nva-CHO, Ubiquitin Aldehyde, YU101, MP-LLL-VS, LDN-57444, Z-GPFL-CHO, Z-LLL-CHO, lovastatin, α-methyl-clasto-lactacystin-β-lactone, mevinolin, MK-803, NIP-L3VS, NP-LLL-VS, NPI-0052 (salinosporamide A), MLN519 (PS-519), NLVS (trileucine vinyl-sulfone), ritonavir, Ro106-9920, Z-LLF-CHO, Z-LL-B(OH)₂, RRRPRPPYLPR, Tyropeptin A, ZL₃VS, PR-11, PR-39, 0106-9920, Proteasome Inhibitor I, Proteasome Inhibitor II, Proteasome Inhibitor III, Proteasome Inhibitor IV, AdaAhx3L3VS, efrapeptin, MG-132, MG-262, MG-115, α-methylomuralide, MG-101, epoxomicin, omuralide, lactacystin and/or NEOSH101. In a specific example, the first agent is SNDX-275 and the second agent is bortezomib. In another embodiment, the first agent is SAHA and the second agent is bortezomib.

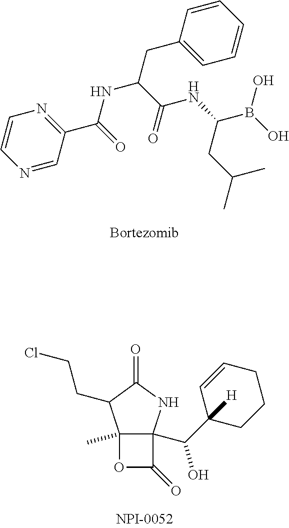

In certain embodiments of the present invention, there is provided a method of treating cancer by administering a first agent to a patient, wherein the first agent sensitizes the cancer to the second agent, which is subsequently administered. In some embodiments, the first agent is SNDX-275 and the second agent is bortezomib.

In other embodiments of the present invention, a method of treating cancer with a first agent and a second agent further comprises administering an additional cancer therapy to a patient. In certain embodiments, the additional cancer therapy is, by way of non-limiting example, surgery, radiation therapy or at least one chemotherapeutic agent. The at least one chemotherapeutic agent includes, by way of non-limiting example, one or more of adriamycin, gemcitabine, mitomycin C, cisplatin, carboplatin, oxaliplatin, fluorouracil, leucovorin, cytarabine, etoposide, capecitabine, temozolomide, doxorubicin, daunomycin, daunorubicin, paclitaxel, docetaxel, cyclophosphamide, ifosfamide, methotrexate, bevacizumab, and trastuzumab.

In some embodiments the pharmaceutical compositions are for the treatment of disorders in a mammal. In specific embodiments, the mammal is a human. In a more specific embodiment, the human is an adult human. In various embodiments, the adult human is more than about 12 years old, more than about 16 years old or more than about 18 years old.

In some embodiments, the first and second agents are administered sequentially. In certain embodiments, the first agent is administered to a patient first and the second agent is administered at a later time or date. In other embodiments, the first and second agents are administered simultaneously. In one embodiment, the first and second agents are administered simultaneously and the second agent is administered again, in the absence of the first agent, at a later time or date. In yet another embodiment, the first agent is administered, in the absence of the second agent, and the second agent is administered together with the first agent at a later time or date. In some embodiments, the first agent is administered as a first pharmaceutical composition and the second agent is administered as a second pharmaceutical composition. In other embodiments, the first and second agents are co-administered in a single pharmaceutical composition.

In various embodiments, the amount of first agent administered is a therapeutically effective amount. In certain embodiments, the therapeutically effective amount of the first agents is about 0.01 to about 1,000 mg/m². In some embodiments, the therapeutically effective amount of the first agent is from about 0.1 to about 500 mg/m². In other embodiments, the therapeutically effective amount of the first agentsis, independently, from about 0.5 to about 100 mg/m². In some embodiments wherein the first agent is SNDX-275, therapeutically effective amounts are about 0.5 to about 15 mg/m². In other embodiments wherein the first agent is SNDX-275, therapeutically effective amounts are about 2 to about 8 mg/m². In specific embodiments wherein the first agent is SNDX-275, the therapeutically effective amount of SNDX-275 is, by way of non-limiting example, about 1, 2, 4, 6 or 8 mg/m². In other embodiments wherein the first agent is MGCD-0103, therapeutically effective amounts are about 5 to about 100 mg/m². In certain embodiments wherein the first agent is MGCD-0103, therapeutically effective amounts are about 10 to about 80 mg/m². In other embodiments wherein the first agent is MGCD-0103, therapeutically effective amounts are about 12 to about 60 mg/m². In still other embodiments wherein the first agent is MGCD-0103, therapeutically effective amounts are about 12.5 to about 36 mg/m². In specific embodiments wherein the first agent is MGCD-0103, the therapeutically effective amount of MGCD-0103 is, by way of non-limiting example, about 5, 10, 12.5, 20, 27, 36, 40, 60, 75 or 80 mg/m².

In certain embodiments, the first agent is administered in a regimen that is therapeutically effective. In various embodiments, the first agent is administered, by way of non-limiting example, twice daily, once daily, five times a week, four times a week, three times a week, twice a week, once weekly, once every two weeks or once every 6 weeks.

In some embodiments, the amount of the second agent administered is a therapeutically effective amount. In certain embodiments, the therapeutically effective amount of the second agents is about 0.01 to about 1,000 mg/m². In some embodiments, the therapeutically effective amount of the first agent is from about 0.1 to about 500 mg/m². In certain embodiments, the therapeutically effective amount of the second agent is about 0.2 to about 100 mg/m². In some embodiments, the therapeutically effective amount of the second agent is about 0.2 to about 20 mg/m². In certain embodiments wherein the second agent is bortezomib, the therapeutically effective amount is about 0.5 to about 5 mg/m². In other embodiments wherein the second agent is bortezomib, the therapeutically effective amount is about 0.7 to about 3 mg/m². In still other embodiments wherein the second agent is bortezomib, the therapeutically effective amount is about 0.9 to about 1.5 mg/m². In specific embodiments wherein the second agent is bortezomib, the therapeutically effective amount is about 0.3, 0.7, 0.9, 1.0, 1.3, 1.4 or 1.5 mg/m².

In certain embodiments, the second agent is administered in a regimen that is therapeutically effective. In various embodiments, the second agent is administered, by way of non-limiting example, twice daily, once daily, five times a week, four times a week, three times a week, twice a week, once weekly, once every two weeks or once every 6 weeks. In specific embodiments, the second agent is delivered twice weekly. In another specific embodiment, the second agent is either PR-171 or bortezomib and is delivered twice a week. In yet another specific embodiment, the second agent is either PR-171 or bortezomib and is delivered three times a week.

In a specific embodiment, the first agent is administered on days 4-11 and the second agent is administered on days 1, 4, 8 and 11. In another specific embodiment, the first agent is administered on days 4 and 11 and the second agent is administered on days 1, 4, 8 and 11. In still another specific embodiment, the first agent is administered on days 1, 3, 5, 8, 10 and 12 and the second agent is administered on days 1, 4, 8 and 11. In yet another specific embodiment, the first agent is administered on days 3 and 10 and the second agent is administered on days 1, 4, 8 and 11. In still another specific embodiment, the first agent is administered on days 4 and 11 and the second agent is administered on days 1, 4, 8 and 11.

In some embodiments, the first and second agents are initially administered on the same day. In specific embodiments, when the first agent is SNDX-275 and the second agent is bortezomib, they are administered on the same day initially, then follow the independent dosing administration schedule recommended (i.e., bortezomib on days 1 and/or 4 and/or 8 and/or 11 of a 21 day cycle, with SNDX275 administered either weekly (day 1, 8, 15, 22) or every other week (day 1 and 15) of a 21 or 22 day cycle.

In some embodiments, the cancer treated by methods described herein is, by way of non-limiting example, brain cancer, breast cancer, lung cancer, ovarian cancer, pancreatic cancer, prostate cancer, renal cancer, colorectal cancer, leukemia, myeloid leukemia, acute myeloid leukemia (AML), glioblastoma, follicular lymphona, pre-B acute leukemia, chronic lymphocytic B-leukemia, mesothelioma or small cell lung cancer. Additional cancers to be treated with the methods and compositions described herein include hematologic and non-hematologic cancers. Hematologic cancer includes multiple myeloma, leukemias, myelodysplastic syndromes, lymphomas, acute leukemia, acute lymphocytic leukemia (ALL), acute myeloid leukemia (AML)/acute nonlymphocytic leukemia (ANLL), chronic lymphocytic leukemia (CLL) and chronic myelogenous leukemia (CML). Lymphoma further includes Hodgkin's lymphoma and non-Hodgkin's lymphoma, cutaneous t-cell lymphoma (CTCL), pediatric acute leukemia, pediatric acute myeloid leukemia, pediatric acute lymphoid leukemia, juvenile myelomonocytic leukemia (JJMML/JCML), and mantle cell lymphoma (MCL). Non-hematologic cancer includes brain cancer, cancers of the head and neck, lung cancer, breast cancer, cancers of the reproductive system, cancers of the gastro-intestinal system, pancreatic cancer, and cancers of the urinary system, cancer of the upper digestive tract or colorectal cancer, bladder cancer or renal cell carcinoma, and prostate cancer.

In certain embodiments, the cancer is a pediatric cancer. In some embodiments, the pediatric cancer is selected from, by way of non-limiting example, brain cancer, leukemia, myeloid leukemia, acute myeloid leukemia (AML), glioblastoma, follicular lymphoma, pre-B acute leukemia, leukemias, myelodysplastic syndromes, lymphomas, acute leukemia, acute lymphocytic leukemia (ALL), acute myeloid leukemia (AML)/acute nonlymphocytic leukemia (ANLL), chronic myelogenous leukemia (CML), Hodgkin's lymphoma, non-Hodgkin's lymphoma, acute leukemia, acute lymphoid lekemia, juvenile myelomonocytic leukemia (JJMML/JCML), cancers of the reproductive system and cancers of the urinary system.

In some embodiments, the cancers to treat with the methods and compositions described herein include cancers that are epithelial malignancies (having epithelial origin), and any cancers (tumors) that express EGFR. Non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer. In some embodiments, the cancer is a pediatric cancer selected from dysplasias and xeroderma pigmentosum.

Additional histone deacetylase mediated cancers which are treated in various embodiments of the present invention include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. These cancers further include cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, metastatic melanoma, basal cell carcinoma, squamous cell carcinoma, other non-epithelial skin cancer; uterine or cervical cancer; uterine corpus cancer; ovarian, vulvar, vaginal, or other female genital cancers; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic. In certain embodiments, the cancer is a pediatric cancer selected from, by way of non-limiting example, cancers of bones and joints, cancers of soft tissue, cancers of genital system, cancers of the eye and orbit, cancers of the nervous system, cancers of the lyphatic system, cancer of the liver, female genital cancers, cancer of the kidney, renal, pelvic or urethral cancer or other cancer of the genito-urinary organs, thyroid cancer or other endocrine cancer.

Yet other histone deacetylase mediated disorder cancers which may be treated using the compositions, combinations and methods described herein include: adenocarcinoma, angiosarcoma, astrocytoma, acoustic neuroma, anaplastic/high-grade astrocytoma, atypical teratoid/rhabdoid tumor, basal cell carcinoma, blastoglioma, chondrosarcoma, choriocarcinoma, chordoma, clear cell sarcoma of the kidney, clear cell sarcoma of the ovary, craniopharyngioma, cutaneous melanoma, cystadenocarcinoma, desmoplastic small round cell tumor, endotheliosarcoma, embryonal carcinoma, ependymoma, Ewing's family of tumors/peripheral neuroepithelioma, primitive neuroectodermal tumor (PNET), epithelial carcinoma, fibrosarcoma, ganglioglioma, ganglioneuroblastoma, gastric cancer, genitourinary tract cancers, germ cell tumors, non-germinomatous germ cell tumors, glioblastoma multiforme, hemangioblastoma, hepatoblastoma, hepatocellular carcinoma, hepatoma, histiocytosis syndromes, Kaposi's sarcoma, Langerhans cell histiocytosis, large cell carcinoma, leiomyosarcoma, liposarcoma, lymphangiosarcoma, lymphangioendotheliosarcoma, malignant fibrous histiocytoma, medullary thyroid carcinoma, medulloblastoma, meningioma mesothelioma, myelomas, myxosarcoma neuroblastoma, neuroblastoma, neurofibrosarcoma, ocular melanoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdoid tumor of the kidney, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, small cell lung cancer and Wilm's tumor. In certain embodiments, the cancer is a pediatric cancer selected from, by way of non-limiting example, angiosarcoma, astrocytoma, acoustic neuroma, anaplastic/high-grade astrocytoma, atypical teratoid/rhabdoid tumor, chondrosarcoma, choriocarcinoma, chordoma, clear cell sarcoma of the kidney, clear cell sarcoma of the ovary, craniopharyngioma, desmoplastic small round cell tumor, endotheliosarcoma, embryonal carcinoma, ependymoma, Ewing's family of tumors/peripheral neuroepithelioma, primitive neuroectodermal tumor (PNET), fibrosarcoma, ganglioglioma, ganglioneuroblastoma, genitourinary tract cancers, germ cell tumors, non-germinomatous germ cell tumors, glioblastoma multiforme, hemangioblastoma, hepatoblastoma, hepatocellular carcinoma, histiocytosis syndromes, Langerhans cell histiocytosis, leiomyosarcoma, liposarcoma, lymphangiosarcoma, lymphangioendotheliosarcoma, malignant fibrous histiocytoma, medullary thyroid carcinoma, medulloblastoma, neuroblastoma, neurofibrosarcoma, oligodendroglioma, osteogenic sarcoma, retinoblastoma, rhabdoid tumor of the kidney, rhabdomyosarcoma, thyroid cancer, and Wilm's tumor.

### Pharmaceutical Compositions and Formulations

The first and second agents of the present invention are each administered individually or in combination. When administered separately from the second agent, the first agent is administered either alone or as a pharmaceutical composition. Likewise, when administered separately from the first agent, the second agent is administered either alone or as a pharmaceutical composition. When the first and second agents are administered in combination, they are administered without any additional components or with additional components in a pharmaceutical composition. In certain embodiments, the pharmaceutical compositions are prepared by admixing at least one active ingredient together with one or more carriers, excipients, buffers, adjuvants, stabilizers, or other materials well known to those skilled in the art and optionally other therapeutic agents. The formulations may conveniently be presented in unit dosage form and may be prepared by any known methods. All formulations and pharmaceutical compositions, as well as any methods of using such pharmaceutical compositions, disclosed herein are contemplated and considered to be within the scope of the disclosure provided herein.

Administration of the agents and pharmaceutical compositions described herein can be effected by any method that enables delivery of the agents to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the agent, and other suitable methods. For example, agents and pharmaceutical compositions described herein can be administered locally to the area in need of treatment. Administration is achieved by, by way of non-limiting example, local infusion during surgery, topical application e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue. Those of ordinary skill in the art are familiar with formulation and administration techniques that can be employed with the agents and methods of the invention, e.g., as discussed in Goodman and Gilman, The Pharmacological Basis of Therapeutics (current edition); Pergamon; and Remington's, Pharmaceutical Sciences (current edition), Mack Publishing Co., Easton, Pa.

The pharmaceutical compositions included herein are those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrasternal), intraperitoneal, transmucosal, transdermal, rectal and topical (including dermal, buccal, sublingual, intranasal, intraocular, and vaginal) administration. The most suitable mode of administration is determined based on the condition of the patient and the specific disorder targeted. In certain embodiments, the pharmaceutical compositions described herein are conveniently formulated in unit dosage form and may be prepared by any of the methods well known in the art of pharmacy. All methods include the step of bringing into association an agent (“active ingredient”) or combination of agents (“active ingredients”) with the carrier which constitutes one or more accessory ingredients. In general, formulations are prepared by uniformly and intimately bringing into association the active ingredient with liquid carriers or finely divided solid carriers or both and then, if necessary, shaping the product into the desired formulation.

In certain embodiments, formulations suitable for oral administration are presented as discrete units such as capsules, cachets or tablets each containing a predetermined amount of the active ingredient or ingredients; as a powder or granules; as a solution or a suspension in an aqueous liquid or a non-aqueous liquid; or as an oil-in-water liquid emulsion or a water-in-oil liquid emulsion. In other embodiments, the active ingredient or ingredients are presented as a bolus, electuary or paste.

In some embodiments, formulations suitable for oral administration include tablets, push-fit capsules made of gelatin, as well as soft, sealed capsules made of gelatin and a plasticizer, such as glycerol or sorbitol. In certain embodiments, tablets are made by compression or molding, optionally with one or more accessory ingredients. In some embodiments, compressed tablets are prepared by compressing in a suitable machine the active ingredient in a free-flowing form such as a powder or granules, optionally mixed with binders (e.g., povidone, gelatin, hydroxypropylmethyl cellulose), inert diluents, preservative, disintegrant (e.g., sodium starch glycolate, cross-linked povidone, cross-linked sodium carboxymethyl cellulose) or lubricating, surface active or dispersing agents. In other embodiments, molded tablets are made by molding in a suitable machine a mixture of the powdered active ingredient or ingredients moistened with an inert liquid diluent. The tablets are optionally coated or scored. In certain embodiments, tablets are formulated so as to provide slow or controlled release of the active ingredient therein. Tablets are optionally provided with an enteric coating, to provide release in parts of the gut other than the stomach. All formulations for oral administration should be in dosages suitable for such administration. In other embodiments, the push-fit capsules contain the active ingredient or ingredients in admixture with filler such as lactose, binders such as starches, and/or lubricants such as talc or magnesium stearate and, optionally, stabilizers. In certain embodiments, soft capsules contain the active ingredient or ingredients dissolved or suspended in suitable liquids, such as fatty oils, liquid paraffin, or liquid polyethylene glycols. In addition, stabilizers are optionally added. Dragee cores are provided with suitable coatings. For this purpose, concentrated sugar solutions may be used, which optionally contains gum arabic, talc, polyvinyl pyrrolidone, carbopol gel, polyethylene glycol, and/or titanium dioxide, lacquer solutions, and suitable organic solvents or solvent mixtures. Dyestuffs, pigments or other color agents are optionally to the tablets or Dragee coatings for identification (e.g., as a pharmaceutical composition comprising the first agent, the second agent or a combination of first and second agents) or to characterize different doses.

In other embodiments, pharmaceutical compositions are formulated for parenteral administration by injection, e.g., by bolus injection or continuous infusion. In various embodiments, formulations for injection are presented in unit dosage form, e.g., in ampoules or in multi-dose containers, with an optional preservative. In certain embodiments, formulations take forms including, by way of non-limiting example, suspensions, solutions or emulsions in oily or aqueous vehicles, and optionally contain formulatory agents such as suspending, stabilizing and/or dispersing agents. In some embodiments, the formulations are presented in unit-dose or multi-dose containers, for example sealed ampoules and vials. In some embodiments, the formulations are stored in powder form or in a freeze-dried (lyophilized) condition requiring only the addition of the sterile liquid carrier, for example, saline or sterile pyrogen-free water, immediately prior to use. Extemporaneous injection solutions and suspensions are prepared, by way of non-limiting example, from sterile powders, granules and tablets of the kind previously described.

Formulations for parenteral administration include aqueous and non-aqueous (oily) sterile injection solutions of the active agents which may contain antioxidants, buffers, biocide, bacteriostats and solutes which render the formulation isotonic with the blood of the intended recipient; and aqueous and non-aqueous sterile suspensions which optionally include suspending agents and thickening agents. Examples of suitable isotonic vehicles for use in such formulations include Sodium Chloride Injection, Ringer's Solution, or Lactated Ringer's Injection. Suitable lipophilic solvents or vehicles include fatty oils such as sesame oil, or synthetic fatty acid esters, such as ethyl oleate or triglycerides, or liposomes or other microparticulate systems may be used to target the agent to blood components or one or more organs. The concentration of the active ingredient or ingredients in the solution varies depending on intended usage.

As such, the invention further provides pharmaceutical compositions and methods of making said pharmaceutical composition. In some embodiments, the pharmaceutical compositions comprise an effective amount of the first and second agents. In other embodiments, a first pharmaceutical composition comprises the first agent and a second pharmaceutical composition comprises the second agent. The pharmaceutical composition may comprise admixing at least one active ingredient with one or more carriers, excipients, buffers, adjuvants, stabilizers, or other materials well known to those skilled in the art and optionally other therapeutic agents. The formulations may conveniently be presented in unit dosage form and may be prepared by any known methods.

Non-limiting examples of excipients that are used in conjunction with the present invention include water, saline, dextrose, glycerol or ethanol. The injectable compositions optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Example of pharmaceutically acceptable carriers that are optionally used include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

In some embodiments, pharmaceutical compositions are formulated as a depot preparation. In certain embodiments, such long acting formulations are administered by implantation (for example subcutaneously or intramuscularly) or by intramuscular injection. Thus, in various examples, the agents or combinations described herein are formulated with suitable polymeric or hydrophobic materials (for example as an emulsion in an acceptable oil) or ion exchange resins, or as sparingly soluble derivatives, for example, as a sparingly soluble salt.

In other embodiments, wherein the pharmaceutical compositions described herein are formulated for buccal or sublingual administration, the pharmaceutical compositions described herein takes the form of tablets, lozenges, pastilles, or gels formulated in conventional manner. Such compositions optionally flavored agents such as sucrose and acacia or tragacanth.

In still other embodiments of the present invention, pharmaceutical compositions are formulated in rectal compositions such as suppositories or retention enemas, e.g., containing conventional suppository bases such as cocoa butter, polyethylene glycol, or other glycerides.

In yet other embodiments, pharmaceutical compositions are administered topically. Topical administration includes non-systemic administration. In certain embodiments, the active ingredient or ingredients are applied externally to the epidermis or the buccal cavity and the instillation of such a agent into the ear, eye and nose, such that the agent does not significantly enter the blood stream. In alternative embodiments, the pharmaceutical compositions described herein are delivered systemically, which includes oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, suspensions, powders, solutions, spray, aerosol, oil, and drops suitable for administration to the eye, ear or nose. In alternative embodiments, a formulation comprises a patch or a dressing such as a bandage or adhesive plaster impregnated with the active ingredient or ingredients and optionally one or more excipients or diluents.

Formulations suitable for topical administration in the mouth include lozenges comprising the active ingredient or ingredients in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient or ingredients are dissolved or suspended in a suitable carrier, including an aqueous solvent.

Formulations for administration by inhalation are conveniently delivered from an insufflator, nebulizer pressurized packs or other convenient means of delivering an aerosol spray. Pressurized packs optionally comprise a suitable propellant such as dichlorodifluoromethane, trichlorofluoromethane, dichlorotetrafluoroethane, carbon dioxide or other suitable gas. In certain aspects of pressurized aerosols, the dosage unit is determined by providing a valve to deliver a metered amount. In alternative embodiments, for administration by inhalation or insufflation, formulations take the form of a dry powder composition, for example a powder mix of the agent and a suitable powder base such as lactose or starch. In certain embodiments, the powder composition is presented in unit dosage form, in for example, capsules, cartridges, gelatin or blister packs from which the powder may be administered with the aid of an inhalator or insufflator.

It should be understood that in addition to the ingredients particularly mentioned above, the agents and compositions described herein may include other agents or components conventional in the art having regard to the type of formulation in question, for example those suitable for oral administration may include flavoring agents.

In certain embodiments, the agents or pharmaceutical compositions described herein are delivered in a vesicle, e.g., a liposome. In various embodiments, the agents and pharmaceutical compositions described herein are delivered in a controlled release system. In one embodiment, a pump is used. In additional embodiments, a controlled release system is placed in proximity of the therapeutic target. In certain aspects of the present invention, the pharmaceutical compositions described are formulated into a formulation suitable for oral use, for example, as tablets, troches, lozenges, aqueous or oily suspensions, dispersible powders or granules, emulsions, hard or soft capsules, or syrups or elixirs. Pharmaceutical compositions intended for oral use are prepared according to any method known to the art for the manufacture of pharmaceutical compositions. In order to provide pharmaceutically elegant and palatable preparations pharmaceutical compositions described herein optionally contain one or more agents selected from the group consisting of sweetening agents, flavoring agents, coloring agents and preserving agents. Tablets contain the active ingredient or ingredients in admixture with one or more non-toxic pharmaceutically acceptable excipient which is suitable for the manufacture of tablets. Excipients include, by way of non-limiting example, inert diluents (e.g., calcium carbonate, sodium carbonate, lactose, calcium phosphate or sodium phosphate), granulating and disintegrating agents (e.g., microcrystalline cellulose, sodium crosscarmellose, corn starch, or alginic acid), binding agents (e.g., starch, gelatin, polyvinyl-pyrrolidone or acacia), and lubricating agents (e.g., magnesium stearate, stearic acid or talc). The tablets are optionally coated or un-coated. Coating of a tablet is accomplished by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. In alternative embodiments, formulations for oral use are in the form of hard gelatin capsules wherein the active ingredient or ingredients are mixed with an inert solid diluent. Suitable inert solid diluents include, by way of non-limiting example, calcium carbonate, calcium phosphate or kaolin. In further embodiments, formulations for oral use are in the form of soft gelatin capsules wherein the active ingredient or ingredients are mixed with water soluble carrier. Water soluble carriers include, by way of non-limiting example, polyethyleneglycol or an oil medium (e.g., peanut oil, liquid paraffin, or olive oil).

Aqueous suspensions contain the active material in admixture with one or more excipient suitable for the manufacture of aqueous suspensions. Suitable excipients include, by way of non-limiting example, suspending agents (e.g., sodium carboxymethylcellulose, methylcellulose, hydroxypropylmethyl-cellulose, sodium alginate, polyvinyl-pyrrolidone, gum tragacanth and gum acacia), dispersing or wetting agents (e.g., a naturally-occurring phosphatide such as lecithin, condensation products of an alkylene oxide with fatty acids such as polyoxyethylene stearate, condensation products of ethylene oxide with long chain aliphatic alcohols such as heptadecaethylene-oxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides, such as polyethylene sorbitan monooleate). The aqueous suspensions optionally contain one or more preservatives (e.g., ethyl, or n-propyl p-hydroxybenzoate), one or more coloring agents, one or more flavoring agents, and one or more sweetening agents (e.g., sucrose, saccharin or aspartame).

In various embodiments, oily suspensions are formulated by suspending the active ingredient in, by way of non-limiting example, a vegetable oil (e.g., arachis oil, olive oil, sesame oil or coconut oil), or in mineral oil (e.g., liquid paraffin). The oily suspensions optionally contain a thickening agent (e.g., beeswax, hard paraffin or cetyl alcohol). Sweetening agents such as those set forth above, and flavoring agents are optionally added to provide a palatable oral preparation. Preservatives and/or anti-oxidants (e.g., butylated hydroxyanisol or alpha-tocopherol) are optionally added as well.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional optional excipients include, by way of non-limiting example, sweetening, flavoring, coloring agents and anti-oxidants. Anti-oxidants include ascorbic acid.

In certain embodiments, pharmaceutical compositions are formulated as oil-in-water emulsions. The oily phase is selected from, by way of non-limiting example, vegetable oil (e.g., olive oil or arachis oil), a mineral oil (e.g., liquid paraffin) or mixtures thereof. Suitable emulsifying agents include naturally-occurring phosphatides (e.g., soy bean lecithin), esters or partial esters derived from fatty acids and hexitol anhydrides (e.g., sorbitan monooleate), and condensation products of the said partial esters with ethylene oxide (e.g., polyoxyethylene sorbitan monooleate). The emulsions optionally contain sweetening agents, flavoring agents, preservatives and antioxidants.

Syrups and elixirs are optionally formulated with sweetening agents (e.g., glycerol, propylene glycol, sorbitol or sucrose). Such formulations also optionally contain one or more demulcent, one or more preservative, one or more flavoring agent, one or more coloring agent and/or one or more antioxidant.

In another embodiment, pharmaceutical compositions are in the form of a sterile injectable aqueous solution. Acceptable vehicles and solvents that are employed are, by way of non-limiting example, water, Ringer's solution and isotonic sodium chloride solution. In some embodiments, the sterile injectable preparation is a sterile injectable oil-in-water microemulsion where the active ingredient is dissolved in the oily phase. In an example, the active ingredient is dissolved in a mixture of soybean oil and lecithin. The oil solution is then introduced into a water and glycerol mixture and processed to form a microemulsion. The injectable solutions or microemulsions may be introduced into a patient's blood-stream by local bolus injection. Alternatively, it may be advantageous to administer the solution or microemulsion in such a way as to maintain a constant circulating concentration of the instant active ingredient or ingredients. In order to maintain such a constant concentration, a continuous intravenous delivery device is utilized in some embodiments. An example of such a device is the Deltec CADD-PLUS™ model 5400 intravenous pump. In other embodiments of the present invention, the pharmaceutical compositions is in the form of a sterile injectable aqueous or oleagenous suspension for intramuscular and subcutaneous administration. This suspension is formulated according to the known art using suitable dispersing, wetting agents and/or suspending agents, all of which are discussed herein. In still other embodiments, the sterile injectable preparation is a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In other embodiments of the present invention, pharmaceutical compositions are administered in the form of suppositories for rectal administration of the drug. In some embodiments, these pharmaceutical compositions are prepared by mixing the active ingredient or ingredients with a suitable non-irritating excipient which is solid at ordinary temperatures but liquid at the rectal temperature and will therefore melt in the rectum to release the drug. Such materials include, by way of non-limiting example, cocoa butter, glycerinated gelatin, hydrogenated vegetable oils, mixtures of polyethylene glycols of various molecular weights and fatty acid esters of polyethylene glycol.

In still other embodiments, the pharmaceutical compositions described herein are formulated for topical use, creams, ointments, jellies, solutions or suspensions, etc., containing an agent or a pharmaceutical composition described herein is used. As used herein, topical application(s) include mouth washes and gargles.

In yet other embodiments, pharmaceutical compositions are administered in intranasal form via topical use of suitable intranasal vehicles and delivery devices, or via transdermal routes, using those forms of transdermal skin patches well known to those of ordinary skill in the art. Tansdermal delivery system, include continuous administration of the active ingredient or ingredients.

### Dosage Forms

In certain embodiments, the pharmaceutical compositions described herein are formulated as a form suitable for oral administration, as a tablet, as a capsule, as a cachet, as a pill, as a lozenge, as a powder or as a granule. In some embodiments of the present invention, the pharmaceutical compositions are formulated as sustained release formulations, solutions, liquids, suspensions, for parenteral injection as a sterile solution, suspension or emulsion, for topical administration as an ointment, cream, lotions, sprays, foams, gel or paste, or for rectal or vaginal administration as a suppository or pessary. In certain embodiments, the pharmaceutical compositions are formulated in unit dosage forms suitable for single administration of precise dosages. In certain aspects, the pharmaceutical composition includes a conventional pharmaceutical carrier or excipient and an agent as described herein as an active ingredient. In addition, other medicinal or pharmaceutical agents, carriers, adjuvants, etc. are included.

Exemplary parenteral administration forms include solutions or suspensions of active agents in sterile aqueous solutions, for example, aqueous propylene glycol or dextrose solutions. Such dosage forms are optionally buffered.

Suitable pharmaceutical carriers include inert diluents or fillers, water and various organic solvents. The pharmaceutical compositions optionally contain additional ingredients such as flavorings, binders, excipients and the like. For example, in a specific embodiment, tablets containing various excipients, such as citric acid are employed together with various disintegrants. Disintegrants include, by way of non-limiting example, starch or other cellulosic material, alginic acid and certain complex silicates and with binding agents such as sucrose, gelatin and acacia. Additionally, lubricating agents such as magnesium stearate, sodium lauryl sulfate and talc are optionally used. Other reagents such as an inhibitor, surfactant or solubilizer, plasticizer, stabilizer, viscosity increasing agent, or film forming agent are also optionally added. In certain embodiments, solid compositions of a similar type are employed in soft and hard filled gelatin capsules. In certain embodiments, the pharmaceutical compositions and/or formulations described herein include lactose or milk sugar and high molecular weight polyethylene glycols. When aqueous suspensions or elixirs are desired for oral administration the active ingredient or ingredients are optionally combined with various sweetening or flavoring agents, coloring agents or dyes and, emulsifying agents or suspending agents, together with diluents such as water, ethanol, propylene glycol, glycerin, or combinations thereof.

### Additional Therapeutic Agents

In some embodiments, the first and second agents described herein are administered with one or more additional therapeutic agent. In these embodiments, either or both of the first and second agents described herein can be in a fixed combination with an additional therapeutic agent or a non-fixed combination with an additional therapeutic agent. In other words, in some embodiments, an additional therapeutic agent is combined with the first agent. In other embodiments, an additional therapeutic agent is combined with the second agent. In still other embodiments, the additional therapeutic agent is administered separately from either the first or second agents. In yet other embodiments, the therapeutic agent is formulated with both the first and second agents in a single formulation. In other embodiments, the first agent is formulated into a first pharmaceutical composition that further comprises an additional therapeutic agent and the second agent is formulated into a second pharmaceutical composition that also contains an additional therapeutic agent. In still other embodiments, the first agent is formulated into a first pharmaceutical composition that does not comprise an additional therapeutic agent and the second agent is formulated into a second pharmaceutical composition that does contain an additional therapeutic agent. In yet other embodiments, the first agent is formulated into a first pharmaceutical composition that further comprises an additional therapeutic agent and the second agent is formulated into a second pharmaceutical composition that does not contain an additional therapeutic agent. In certain embodiments, the different pharmaceutical compositions are distinguished by color (e.g., by using different coloring agents in each of the pharmaceutical compositions utilized). Provided below are various embodiments of additional therapeutic agents that are combined with the first and second agents described hereinabove.

As used herein, any reference to an additional therapeutic agent refers to one or more additional therapeutic agents. As such, in one embodiment, provided herein is a method of treating a histone deacetylase mediated disorder with a first agent, a second agent, and an additional therapeutic agent. In another embodiment, provided herein is a method of treating a histone deacetylase mediated disorder with a first agent, a second agent, a first additional therapeutic agent, and a second additional therapeutic agent.

In one embodiment of the present invention, the additional therapeutic agent is an anti-hypertensive agent. In other embodiments of the present invention, the additional therapeutic agent is an agent that enhances the efficacy of either or both of the first and second agents. In still other embodiments, the additional therapeutic agent is another therapeutic agent (including a therapeutic regimen, therapy or treatment) that also has a therapeutic benefit. In various embodiments, the additional therapeutic agent provides an additive benefit. In other embodiments, the additional therapeutic agent provides a synergistic benefit with either one or both of the first and second agents.

Therapies include, but are not limited to, administration of other therapeutic agents, radiation therapy or both. In the instances where the first and/or second agents described herein are administered with other therapeutic agents, the agents described herein need not be administered in the same pharmaceutical composition as any additional therapeutic agents. Furthermore, in various embodiments, the first agent, second agent and any additional therapeutic agent are administered by different routes. In other embodiments, one or more of the first agent, second agent and any additional therapeutic agent is administered by the same route. In still other embodiments, each of the first agent, second agent and any additional therapeutic agent are administered by the same route. In one example, one or more of the agents is administered orally, while one or more of the other agents are administered intravenously. In further embodiments, the dosage, modes of administration and times of administration of one or more of the agents is modified after administration is begun.

In certain embodiments, the first agent, second agent, and where applicable additional therapeutic agents are administered concurrently (e.g., simultaneously, essentially simultaneously or within the same treatment protocol). In other embodiments, the first agent, second agent, and where applicable additional therapeutic agent are administered sequentially. In still other embodiments, certain agents are administered concurrently while others are administered sequentially. The manner in which the agents are delivered depends on the nature of the disease, the condition of the patient, and/or the choice of additional therapeutic agent and/or therapy (e.g., radiation) to be administered. Furthermore, it is to be understood that these administration methods include the administration of one or all of the agents in a pharmaceutical composition as described herein.

In combinational applications and uses, the first agent, second agent and the additional therapeutic agent need not be administered simultaneously or essentially simultaneously. Indeed, in some embodiments, the initial order of administration of the agents or pharmaceutical compositions thereof is not important. Thus, in certain embodiments, the first and second agent or pharmaceutical compositions thereof are administered prior to the administration of the additional therapeutic agent. In another embodiment, the additional therapeutic agent is administered prior to the first and second agents. In still another embodiment, the first agent is administered first, the additional therapeutic agent is administered second, and the second agent is administered third. In various embodiments, a treatment protocol repeats the sequence of steps described or combines them. In certain embodiments, the treatment protocol is repeated until treatment is complete. In further embodiments, as treatment proceeds a treatment protocol is modified according to the individual patient's needs. Indications of the patient's needs include, but are not limited to, relief of disease-related symptoms, inhibition of tumor growth, actual shrinkage of the tumor, or inhibition of metastasis. Tumor size is measured by standard methods, including radiological studies (e.g., CAT or MRI scan).

Specific, non-limiting examples of additional therapeutic agents are found in the pharmacotherapeutic classifications listed below. These lists are illustrative only and are not to be construed as limiting. Moreover, as with the first and second agents, the additional therapeutic agent is administered in any acceptable manner including, by way of non-limiting example, oral, intravenous, intraocular, subcutaneous, dermal, and inhaled topical. As with the first and second agents, the additional therapeutic agent need not be administered in a manner identical to either or both of the first and second agents.

In some embodiments, additional therapeutic agents include chemotherapeutic agents. Non-limiting examples of chemotherapeutic agents are anticancer agents, alkylating agents, cytotoxic agents, antimetabolic agents, hormonal agents, plant-derived agents, and biologic agents.

Anti-tumor substances are selected from, by way of non-limiting example, mitotic inhibitors (e.g., vinblastine), alkylating agents (e.g., cis-platin, carboplatin and cyclophosphamide), anti-metabolites (5-fluorouracil, cytosine arabinside and hydroxyurea), one of the anti-metabolites disclosed in European Patent Application No. 239362 (e.g., N-(5-[N-(3,4-dihydro-2-methyl-4-oxoquinazolin-6-ylmethyl)-N-methylamino]-2-thenoyl)-L-glutamic acid), growth factor inhibitors, cell cycle inhibitors, intercalating antibiotics (e.g, adriamycin and bleomycin), enzymes (e.g., interferon), anti-hormones (e.g., anti-estrogens such as Nolvadex™ (tamoxifen) or anti-androgens such as Casodex™ (4′-cyano-3-(4-fluorophenylsulphonyl)-2-hydroxy-2-methyl-3′-(trifluoromethyl)propionanilide)). As with any treatment regiment described herein, these chemotherapeutic agents are administered, in various embodiments, simultaneous, sequential or separate from either or both of the first and second agents.

Alkylating agents include, by way of non-limiting example, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (e.g., altretamine, dacarbazine, and procarbazine), platinum compounds (e.g., oxaliplatin, carboplastin and cisplatin).

Cytotoxic agents include, by way of non-limiting example, anthracyclines (e.g. doxorubicin, daunorubicin, epirubicin, idarubicin and anthracenedione), mitomycin C, bleomycin, dactinomycin, plicatomycin.

Antimetabolic agents are a group of drugs that interfere with metabolic processes vital to the physiology and proliferation of cancer cells. Antimetabolic agents include, by way of non-limiting example, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), forodesine hydrocloride, clofarabine, asparaginase, and gemcitabine.

Hormonal agents are a group of drug that regulate the growth and development of their target organs. Hormonal agents include sex steroids and their derivatives and analogs thereof, such as estrogens, androgens, and progestins. Hormonal agents include, by way of non-limiting example, synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

Plant-derived agents include, by way of non-limiting example, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis.

As used herein, the phrase “biologic agents” refers to a group of biomolecules that elicit cancer/tumor regression when used alone or in combination with chemotherapy and/or radiotherapy. Biologic agents include, by way of non-limiting example, immuno-modulating proteins such as cytokines, monoclonal antibodies against tumor antigens, tumor suppressor genes, and cancer vaccines.

Furthermore, in various embodiments of the present invention, the additional therapeutic agent (or chemotherapeutic agent) is selected from, by way of non-limiting example, aromatase inhibitors, antiestrogen, anti-androgen, corticosteroids, gonadorelin agonists, topoisomerase 1 and 2 inhibitors, microtubule active agents, alkylating agents, nitrosoureas, antineoplastic antimetabolites, platinum containing compounds, lipid or protein kinase targeting agents, IMiDs, protein or lipid phosphatase targeting agents, anti-angiogenic agents, Akt inhibitors, IGF-I inhibitors, FGF3 modulators, mTOR inhibitors, Smac mimetics, other HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multlikinase inhibitors, bisphosphanates, rapamycin derivatives, anti-apoptotic pathway inhibitors, apoptotic pathway agonists, PPAR agonists, inhibitors of Ras isoforms (e.g., tipifamib, lonafarnib), telomerase inhibitors, protease inhibitors, metalloproteinase inhibitors, aminopeptidase inhibitors, dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, caminomycin, idarubicin, epirubicin, esorubicin, mitoxantrone, bleomycins A, A₂, and B, camptothecin, Irinotecan®, Topotecan®, 9-aminocamptothecin, 10,11-methylenedioxycamptothecin, 9-nitrocamptothecin, bortezomib, temozolomide, TAS 103, NPI0052, combretastatin, combretastatin A-2, combretastatin A-4, calicheamicins, neocarcinostatins, epothilones A B, C, and semi-synthetic variants, Herceptin®, Rituxan®, CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva®, Iressa®, Imatinib®, Miltefosine®, Perifosine®, aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel.

In further embodiments, additional therapeutic agents include interleukin 2 (IL-2), interleukin 4 (IL-4), interleukin-6 (IL-6), and interleukin 12 (IL-12).

Interferons include more than 23 related subtypes with overlapping activities, all of the IFN subtypes within the scope of the present invention. IFN has demonstrated activity against many solid and hematologic malignancies, the later appearing to be particularly sensitive.

Other cytokines included within the scope of the invention are cytokines that exert profound effects on hematopoiesis and immune functions. Examples of such cytokines include, by way of non-limiting example, erythropoietin, granulocyte-CSF (filgrastin), and granulocyte, macrophage-CSF (sargramostim).

Other immuno-modulating agents include, by way of non-limiting example, bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, including tumor-specific antigens. Monoclonal antibodies of the present invention include, by way of non-limiting example, HERCEPTIN® and RITUXAN®.

As used herein, tumor suppressor genes are genes that function to inhibit the cell growth and division cycles, thus preventing the development of neoplasia. Tumor suppressor genes include, by way of non-limiting example, DPC-4, NF-1, NF-2, RB, p53, WT1, BRCA1 and BRCA2.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to the tumor cell, TAAs provide targets for the immune system to recognize and cause their destruction. TAAs include, by way of non-limiting example, gangliosides (GM2), prostate specific antigen (PSA), alpha-fetoprotein (AFP), carcinoembryonic antigen (CEA) (produced by colon cancers and other adenocarcinomas, e.g. breast, lung, gastric, and pancreas cancer s), melanoma associated antigens (MART-1, gp 100, MAGE 1,3 tyrosinase), papillomavirus E6 and E7 fragments, whole cells or portions/lysates of antologous tumor cells and allogeneic tumor cells.

In some embodiments, the additional therapeutic agent is a proteasome inhibitor. Proteasome inhibitors include, by way of non-limiting example, bortezomib (Velcade, PS-341), PR-171, NPI-0052 (salinosporamide A), MG-132, omuralide, lactacystin and NEOSH101. In a specific embodiment, the first and second agents are administered concurrently or sequentially (in either order) and the proteasome inhibitor is administered after both the first and second agents have been administered. In certain embodiments, the proteasome inhibitor is bortezomib.

In certain embodiments, an adjuvant is used in the combination to augment the immune response to TAAs. Examples of adjuvants include, by way of non-limiting example, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan.

In certain embodiments of the present invention, the additional therapeutic agent is used to treat inflammation and/or pain. In various embodiments, the additional therapeutic agent is, by way of non-limiting example, a corticosteroid, a non-steroidal anti-inflammatory agent, a muscle relaxant or combinations thereof. In other embodiments, the additional therapeutic agent is, by way of non-limiting example, an anesthetic, an expectorant, an antidepressant, an anticonvulsant, an antihypertensive, an opioid, a cannabinoid, capsaicin, or combinations thereof.

In other embodiments of the present invention, the additional therapeutic agent is, by way of non-limiting example, betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream (Eutectic Mixture of Local Anesthetics (lidocaine 2.5% and prilocalne 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, inhibitors of inducible nitric oxide synthase; capsaicin or combinations thereof.

In certain embodiments, an additional therapeutic agent is selected from beta-blockers, carbonic anhydrase inhibitors, □- and □-adrenergic antagonists including a1-adrenergic antagonists, □2 agonists, miotics, prostaglandin analogs, corticosteroids, immunosuppressant agents, timolol, betaxolol, levobetaxolol, carteolol, levobunolol, propranolol, brinzolamide, dorzolamide, nipradilol, iopidine, brimonidine, pilocarpine, epinephrine, latanoprost, travoprost, bimatoprost, unoprostone, dexamethasone, prednisone, methylprednisolone, azathioprine, cyclosporine, immunoglobulins, and combinations thereof is administered.

In still other embodiments of the present invention, the first agent, second agent and additional therapeutic agent are utilized in a method for treating autoimmune disorders. In certain embodiments, the additional therapeutic agent is selected from, by way of non-limiting example, corticosteroids, immunosuppressants, prostaglandin analogs and antimetabolites, dexamethasome, prednisone, methylprednisolone, azathioprine, cyclosporine, immunoglobulins, latanoprost, travoprost, bimatoprost, unoprostone, infliximab, rituximab, methotrexate and combinations thereof.

In yet other embodiments of the present invention, the first agent, second agent and additional therapeutic agent are utilized in a method for treating metabolic disorders. In certain embodiments, the additional therapeutic agent is selected from, by way of non-limiting example, insulin, insulin derivatives and mimetics, insulin secretagogues, insulin sensitizers, biguanide agents, alpha-glucosidase inhibitors, insulinotropic sulfonylurea receptor ligands, protein tyrosine phosphatase-1B (PTP-1B) inhibitors, GSK3 (glycogen synthase kinase-3) inhibitors, GLP-1 (glucagon like peptide-1), GLP-1 analogs, DPPIV (dipeptidyl peptidase IV) inhibitors, RXR ligands sodium-dependent glucose co-transporter inhibitors, glycogen phosphorylase A inhibitors, an AGE breaker, PPAR modulators, non-glitazone type PPARS agonist, tformin, Glipizide, glyburide, Amaryl, meglitinides, nateglinide, repaglinide, PT-112, SB-517955, SB4195052, SB-216763, N,N-57-05441, N,N-57-05445, GW-0791, AGN-.sup.194.sup.204, T-1095, BAY R3401, acarbose Exendin-4, DPP728, LAF237, vildagliptin, MK-0431, saxagliptin, GSK23A, pioglitazone, rosiglitazone, (R)-1-{4-[5-methyl-2-(4-trifluoromethyl-phenyl)-oxazol-4-ylmethoxy]-benzenesulfonyl}2,3-dihydro-1H-indole-2-carboxylic acid, GI-262570 and combinations thereof.

In some embodiments of the invention, the treatments and uses described herein carry with them side effects that include, for example, nausea, vomiting, immunosuppression and susceptibility to infections, anemia and pain. Therefore, in certain embodiments, the additional therapeutic agent is any agent that abrogates treats, reduces the incidence of or prevents such side effects. In some embodiments, additional therapeutic agents include, by way of non-limiting example, anti-emetic agents, immuno-restorative agents, antibiotic agents, anemia treatment agents, and analgesic agents for treatment of pain and inflammation.

As used herein anti-emetic agents are defined as drugs effective for treatment of nausea and emesis (vomiting). Anti-emetic agents include, by way of non-limiting example, 5-HT₃ seratonin receptor antagonists. 5-HT₃ antagonists include, by way of non-limiting example, dolasetron (Anzemet®), granisetron (Kytril®), ondansetron (Zofran®), palonosetron and tropisetron. Other anti-emetic agents include, by way of non-limiting example, dopamine receptor antagonists (e.g., chlorpromazine, domperidone, droperidol, haloperidol, metaclopramide, promethazine, and prochlorperazine), antihistamines (e.g., cyclizine, diphenhydramine, dimenhydrinate, meclizine, promethazine, and hydroxyzine), lorazepram, scopolamine, dexamethasone, Emetrol®, propofol, and trimethobenzamide.

As used herein, immuno-restorative agents are defined as drugs that counter the immuno-suppressive effects of cancer therapies. Immuno-restorative agents include, by way of non-limiting example, synthetic analogs of the hormone, granulocyte colony stimulating factor (G-CSF), filgrastim (Neupogen®), PEG-filgrastim (Neulasta®) and lenograstim.

As used herein, antibiotic agents include drugs that have anti-bacterial, anti-fungal, and anti-parasite properties. Antibiotics include, by way of non-limiting example, amikacin, gentamicin, kanamycin, neomycin, netilmicin, streptomycin, tobramycin, loracarbef, ertapenem, cilastatin, meropenem, cefadroxil, cefazolin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicoplanin, vancomycin, azithromycin, clarithromycin, dirithromycin, erthromycin, roxithromycin, troleandomycin, aztreonam, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, benzolamide, bumetanide, chlorthalidone, clopamide, dichlorphenamide, ethoxzolamide, indapamide, mafenide, mefruside, metolazone, probenecid, sulfanilamides, sulfamethoxazole, sulfasalazine, sumatriptan, xipamide, democlocycline, doxycycline, minocycline, oxytetracycline, tetracycline, chloramphenical, clindamycin, ethambutol, fosfomycin, fusidic acid, furazolidone, isoniazid, linezolid, metronidazole, mupirocin, nitrofurantoin, platesimycin, pyrazinamide, dalfopristin, rifampin, spectinomycin, and telithromycin.

As used herein, anemia treatment agents are drugs directed toward treatment of low red blood cell and platelet production. Anemia treatment agents include, by way of non-limiting example, recombinant erythropoietin (EPOGEN®, Dynopro®) and Darbepoetin alfa (Aranesp®).

In further embodiments, the additional therapeutic agent is selected from, by way of non-limiting example, corticosteroids, non-steroidal anti-inflammatories, muscle relaxants, anesthetics, expectorants, antidepressants, anticonvulsants, antihypertensives, opioids, topical cannabinoids, and capsaicin.

It is noted that any reference to the administration of a first agent, a second agent, an additional therapeutic agent, or any combination thereof includes the administration of a pharmaceutical composition comprising the agent or agents disclosed as being administered.

### Kits

The agents, pharmaceutical compositions and methods described herein provide kits for the treatment of disorders, such as the ones described herein. These kits comprise a first and second agent or pharmaceutical compositions thereof in a container and, optionally, instructions teaching the use of the kit according to the various methods and approaches described herein. Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits are, in some embodiments, marketed directly to the consumer. In certain embodiments, the packaging material further comprises a container for housing the composition and optionally a label affixed to the container. The kit optionally comprises additional components, such as, by way of non-limiting example, syringes for administration of the composition.

In one embodiment of the present invention, the kit described herein contains a therapeutically effect amount of first agent and a therapeutically effective amount of a second agent, wherein the first and second agents are as described herein. In certain embodiments, the present invention provides for a kit comprising a pharmaceutical composition, wherein the pharmaceutical composition contains either one of or both of the first and second agents.

In another embodiment, the kit comprises at least one first pharmaceutical composition and at least one second pharmaceutical composition. The first pharmaceutical composition contains a therapeutically effective amount of the first agent and the second pharmaceutical composition contains a therapeutically effective amount of the second agent. In some embodiments of the present invention, the first pharmaceutical composition does not contain a therapeutically effective amount of the second agent. In certain embodiments, the second pharmaceutical composition does not contain a therapeutically effective amount of the first agent. In other embodiments, the first pharmaceutical composition contains a therapeutically effective amount of the first agent and a therapeutically effective amount of the second agent. In still other embodiments, the second pharmaceutical composition contains a therapeutically effective amount of the second agent and a therapeutically effective amount of the first agent. In some embodiments, the kit further contains a third pharmaceutical composition that contains therapeutically effective amounts of the first and second agents.

In a specific embodiment, the kit comprises (1) a first pharmaceutical composition that contains a therapeutically effective amount of a first agent and a therapeutically effective amount of the second agent, and (2) a second pharmaceutical composition that contains a therapeutically effective amount of the second agent and does not contain a therapeutically effective amount of the first agent. In another specific embodiment, the kit comprises (1) a first pharmaceutical composition that contains a therapeutically effective amount of a first agent and does not contain a therapeutically effective amount of the second agent, and (2) a second pharmaceutical composition that contains a therapeutically effective amount of the second agent and a therapeutically effective amount of the first agent.

In some embodiments, the kit comprises a first pharmaceutical composition that is visibly different from a second pharmaceutical composition. The visible differences may be for example shape, size, color, state (e.g. liquid/solid), physical markings (e.g. letters, numbers) and the like. In certain embodiments, the kit comprises a first pharmaceutical composition that is a first color and a second pharmaceutical composition that is a second color. In embodiments wherein the first and second colors are different, the different colors of the first and second pharmaceutical compositions is used, e.g., to distinguish between the first and second pharmaceutical compositions. In further embodiments, the third pharmaceutical composition is a third color.

In some embodiments, wherein the packaging material further comprises a container for housing the pharmaceutical composition, the kit comprises a first pharmaceutical composition that is in a different physical location within the kit from a second pharmaceutical composition. In some embodiments, the different physical locations containing the first and second pharmaceutical compositions comprise separately sealed individual compartments. In certain embodiments, the kit comprises a first pharmaceutical composition that is in a first separately sealed individual compartment and a second pharmaceutical composition that is in a second separately sealed individual compartment. In embodiments wherein the first and second compartments are separate, the different locations of the first and second pharmaceutical compositions are used, e.g., to distinguish between the first and second pharmaceutical compositions. In further embodiments, the third pharmaceutical composition is in a third physical location within the kit.

The use of the term “agents” is understood to refer to either the first and second agent or the first, second and additional therapeutic agent.

### EXAMPLES

Below are provided non-limiting examples of the present invention:

### Example 1

### Evaluation of Synergistic Effect in Colorectal Carcinoma

The following is an example the evaluation of the synergistic effect of a first HDAC inhibitor in combination with a proteasome inhibitor in colorectal carcinoma (CRC) in vivo. The activity of a first HDAC inhibitor as single agent and in combination with a proteasome inhibitor is evaluated in nude mice bearing CRC cell lines. Mice bearing CRC tumors are randomly assigned to treatment groups, and the effect of SNDX-275, bortezomib, and an SNDX-275/bortezomib combination on tumor growth is evaluated. Nude mice are implanted with CRC cell-lines. Implantation of a tumor is achieved through established tumor transplantation techniques (e.g., injection or surgical orthotopic implantation). Upon establishment of the CRC tumor, as determined by tumor volume measurement, the effect of SNDX-275, bortezomib, and a combination of SNDX-275 and bortezomib is evaluated for inhibition of tumor growth. Each agent (SNDX-275, bortezomib, or SNDX-275/bortezomib combination) is administered to different groups of mice in different dosages. Each agent is administered as follows: SNDX-275—2 doses, bortezomib—2 doses, SNDX-275/bortezomib combination—4 doses. Biopsies and measurements of the tumors are taken at 4 time points corresponding to 0, 48, 72, and 96 hours post-treatment. Tumor volumes are measured for each time point to determine efficacy of the agents.

### Example 2

### Evaluation of Synergistic Effect in Breast Cancer

The following is an example the evaluation of the synergistic effect of an HDAC inhibitor in combination with a proteasome inhibitor in breast cancer in vivo. The activity of a first HDAC inhibitor as single agent and in combination with a proteasome inhibitor is evaluated in nude mice bearing breast cancer cell lines. Mice bearing breast cancer tumors are randomly assigned to treatment groups, and the effect of SNDX-275, bortezomib, and an SNDX-275/bortezomib combination on tumor growth is evaluated. Nude mice are implanted with breast cancer cell lines. Implantation of a tumor is achieved through established tumor transplantation techniques (e.g., injection or surgical orthotopic implantation). Upon establishment of the breast cancer tumor, as determined by tumor volume measurement, the effect of SNDX-275, bortezomib, and a combination of SNDX-275 and bortezomib is evaluated for inhibition of tumor growth. Each agent (SNDX-275, bortezomib, or SNDX-275/bortezomib combination) is administered to different groups of mice in different dosages. Each agent is administered as follows: SNDX-275—2 doses, bortezomib—2 doses, SNDX-275/bortezomib combination—4 doses. Biopsies and measurements of the tumors are taken at 4 time points corresponding to 0, 48, 72, and 96 hours post-treatment. Tumor volumes are measured for each time point to determine efficacy of the agents.

### Example 3

### Treatment with SNDX-275 and Bortezomib

Human Clinical Trial of the Safety and/or Efficacy of SNDX-275/Bortezomib Combination Therapy

### Objective:

To compare the safety and pharmacokinetics of administered SNDX-275 and bortezomib.

Study Design:

This will be a Phase I, single-center, open-label, randomized dose escalation study followed by a Phase II study in cancer patients with disease that can be biopsied (e.g., breast cancer, non-small cell lung cancer, prostate cancer, pancreatic cancer, colorectal cancer, head and neck cancer). Patients should not have had exposure to SNDX-275 or bortezomib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I:

Patients receive oral bortezomib on days 1 and/or 4, and/or 8, and/or 11, and oral SNDX-275 on days 1, 8, and 15 or days 1 and 15. Doses of either bortezomib or SNDX-275 may be held or modified for toxicity based on assessments as outlined below. Treatment repeats every 28 days in the absence of unacceptable toxicity. Cohorts of 3-6 patients receive escalating doses of bortezomib and SNDX-275 until the maximum tolerated dose (MTD) for the combination of bortezomib and SNDX-275 is determined. Test dose ranges are initially determined via the established individual dose ranges for SNDX-275 and bortezomib. A standard dosage for bortezomib is 1-1.3 mg/m² per dose. An established dosage for SNDX-275 includes 2-4 mg/m² per dose. Additional dosages, both decreasing and increasing in amount as well as frequency, are determined based on the standard dose for both SNDX-275 and bortezomib. The MTD is defined as the dose preceding that at which 2 or more of 3 to 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II:

Patients receive bortezomib as in phase I at the MTD determined in phase I and SNDX-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they continue to meet original eligibility criteria.

Blood Sampling:

Venous blood samples (5 mL) for determination of serum concentrations are obtained at about 10 minutes prior to dosing and at several time points on days 1, 2, 4, 8, 11, 15, 29, and 42 following dosing, and at the end of study participation. Each serum sample is divided into two aliquots. All serum samples are stored at −20° C. Serum samples are shipped on dry ice.

Pharmacokinetics:

Patients undergo plasma/serum sample collection for pharmacokinetic evaluation before beginning treatment and at days 1, 2, 4, 8, 11, 15, 29, and 42 following dosing, and at the end of study participation. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to Combination Therapy:

Patient response is assessed via imaging with X-ray, bone scan, CT scans, PET scans, PET/CT scans, and/or magnetic resonance imaging (MRI). Imaging is performed prior to beginning the study and at the end of the first cycle, with additional imaging performed every six weeks or at the end of subsequent cycles. Imaging modalities are chosen based upon the cancer type and feasibility/availability, and the same imaging modality is utilized throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients may also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 4

### Treatment with SNDX-275 and PR-171

Human Clinical Trial of the Safety and/or Efficacy of SNDX-275/PR-171 Combination Therapy

### Objective:

To compare the safety and pharmacokinetics of administered SNDX-275 and PR-171.

Study Design:

This will be a Phase I, single-center, open-label, randomized dose escalation study followed by a Phase II study in cancer patients with disease that can be biopsied (e.g., breast cancer, non-small cell lung cancer, prostate cancer, pancreatic cancer, colorectal cancer, head cancer and neck cancer). Patients should not have had exposure to SNDX-275 or PR-171 prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I:

Patients receive intravenous PR-171 on days 1-5 and 8-12 and oral SNDX-275 on days 1, 8, and 15 or 1 and 15. Treatment repeats every 28 days in the absence of unacceptable toxicity. Cohorts of 3-6 patients receive escalating doses of PR-171 and SNDX-275 until the maximum tolerated dose (MTD) for the combination of PR-171 and SNDX-275 is determined. Test dose ranges are initially determined via the established individual dose ranges for SNDX-275 and PR-171. A standard dosage for PR-171 is between 1.2 mg/m² and 15 mg/m² QD×5 on a two week cycle with 9 days rest. An established dosage for SNDX-275 includes 2-4 mg/m² per dose. Additional dosages, both decreasing and increasing in amount as well a frequency, are determined based on the standard dose for both SNDX-275 and PR-171. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II:

Patients receive PR-171 as in phase I at the MTD determined in phase I and SNDX-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling

Serial blood is drawn by direct vein puncture before and after administration of SNDX-275 or PR-171. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics:

Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to Combination Therapy:

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 5

### Treatment with MGCD-0103 and Bortezomib

Human Clinical Trial of the Safety and/or Efficacy of MGCD-0103/Bortezomib combination therapy

### Objective:

To compare the safety and pharmacokinetics of administered MGCD-0103 and bortezomib.

Study Design:

This will be a Phase I, single-center, open-label, randomized dose escalation study followed by a Phase II study in cancer patients with disease that can be biopsied (e.g., breast cancer, non-small cell lung cancer, prostate cancer, pancreatic cancer, colorectal cancer, head and neck cancer). Patients should not have had exposure to MGCD-0103 or bortezomib prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I:

Patients receive oral bortezomib on days 1 and/or 4 and/or 8 and/or 11 and oral MGCD-0103 on days 1-14 every 21 days, or oral MGCD-0103 given three times weekly for 2 weeks, every 21 days. Treatment repeats every 21-28 days in the absence of unacceptable toxicity. Cohorts of 3-6 patients receive escalating doses of bortezomib and MGCD-0103 until the maximum tolerated dose (MTD) for the combination of bortezomib and MGCD-0103 is determined. Test dose ranges are initially determined via the established individual dose ranges for MGCD-0103 and bortezomib. A standard dosage for bortezomib is 1-1.3 mg/m² twice a week for two weeks given every 21 days. An established dosage for MGCD-0103 includes 12.5 mg/m2/day on days 1-14 every 21 days, or oral MGCD-0103 12.5-36 mg/m2/day, given three times weekly for 2 weeks, every 21 days. Additional dosages, both decreasing and increasing in amount as well a frequency, are determined based on the standard dose for both MGCD-0103 and bortezomib. The MTD is defined as the dose preceding that at which 2 or more of 3 to 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II:

Patients receive bortezomib as in phase I at the MTD determined in phase I and MGCD-0103 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling

Venous blood samples (5 mL) for determination of serum concentrations are obtained at about 10 minutes prior to dosing and at several time points on days 1, 2, 4, 8, 11, 15, 29, and 42 following dosing, and at the end of study participation. Each serum sample is divided into two aliquots. All serum samples are stored at −20° C. Serum samples are shipped on dry ice.

Pharmacokinetics:

Patients undergo plasma/serum sample collection for pharmacokinetic evaluation before beginning treatment and at days 1, 2, 4, 8, 11, 15, 29, and 42 following dosing, and at the end of study participation. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to Combination Therapy:

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 6

### Treatment with MGCD-0103 and PR-171

Human Clinical Trial of the Safety and/or Efficacy of MGCD-0103/PR-171 Combination Therapy

### Objective:

To compare the safety and pharmacokinetics of administered MGCD-0103 and PRI-171.

### Study Design:

This will be a Phase I, single-center, open-label, randomized dose escalation study followed by a Phase II study in cancer patients with disease that can be biopsied (e.g., breast cancer, non-small cell lung cancer, prostate cancer, pancreatic cancer, colorectal cancer, head and neck cancer). Patients should not have had exposure to MGCD-0103 or PR-171 prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I:

Patients receive intravenous PR-171 on days 1-5 and 8-10 and oral MGCD-0103 on days 1-14 every 21 days, or oral MGCD-0103 given three times weekly for 2 weeks, every 21 days. Treatment repeats every 28 days in the absence of unacceptable toxicity. Cohorts of 3-6 patients receive escalating doses of PR-171 and MGCD-0103 until the maximum tolerated dose (MTD) for the combination of PRI-171 and MGCD-0103 is determined. Test dose ranges are initially determined via the established individual dose ranges for MGCD-0103 and PR-171. A standard dosage for PR-171 is between 1.2 mg/m² and 15 mg/m² QD×5 on a two week cycle with 9 days rest. An established dosage for MGCD-0103 includes 12.5 mg/m2/day on days 1-14 every 21 days, or oral MGCD-0103 12.5-36 mg/m2/day, given three times weekly for 2 weeks, every 21 days. Additional dosages, both decreasing and increasing in amount as well a frequency, are determined based on the standard dose for both MGCD-0103 and PR-171. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II:

Patients receive PR-171 as in phase I at the MTD determined in phase I and MGCD-0103 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling

Serial blood is drawn by direct vein puncture before and after administration of MGCD-0103 or PR-171. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics:

Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to Combination Therapy:

Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92 (3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

### Example 7

### Parenteral Composition

An i.v. solution is prepared in a sterile isotonic solution of water for injection and sodium chloride (˜300 mOsm) at pH 11.2 with a buffer capacity of 0.006 mol/l/pH unit. The protocol for preparation of 100 ml of a 5 mg/ml a first and/or second agent for i.v. infusion is as follows: add 25 ml of NaOH (0.25 N) to 0.5 g of a first and/or second agent and stir until dissolved without heating. Add 25 ml of water for injection and 0.55 g of NaCl and stir until dissolved. Add 0.1N HCl slowly until the pH of the solution is 11.2. The volume is adjusted to 100 ml. The pH is checked and maintained between 11.0 and 11.2. The solution is subsequently sterilized by filtration through a cellulose acetate (0.22 μm) filter before administration.

### Example 8

### Oral Composition

A pharmaceutical composition for oral delivery is prepared by mixing 100 mg of a first and/or second agent with 750 mg of a starch. The mixture is incorporated into an oral dosage unit, such as a hard geletin capsule or coated tablet, which is suitable for oral administration.

Many modifications, equivalents, and variations of the present invention are possible in light of the above teachings, therefore, it is to be understood that within the scope of the appended claims, the invention may be practiced other than as specifically described.

### Example 9

### Broad Spectrum Anti-Tumor Activity

Cell lines derived from patients with acute lymphoblastic leukemia (ALL), acute myelogenous leukemia (AML), rhabdomyosarcoma (RMS), Ewing's sarcoma (EWS), or osteosarcoma (OS) are treated with SAHA (vorinostat), SNDX-275 (entinostat), or bortezomib for 48 hours and relative cell numbers are determined by MTT assay. IC₅₀ values are calculated by non-linear regression. MLL lines are mixed lineage leukemias. FIGS. 1, 2A, and 2B illustrate the results. Mean values and standard errors of FIG. 1 are derived from 4-7 independent experiments.

### Example 10

### Induction of Cell Death

Cultures of the indicated cell lines are exposed to SAHA, SNDX-275, or vehicle only (DMSO) for 48 hours. Apoptotic and dead cells are identified by flow cytometric analysis of cells stained with propodium iodide and YO-PRO®-1-iodide. Cells undergoing apoptosis are stained with YO-PRO-1, but are impermeable to propidium iodide. Dead cells and cells in late apoptosis are permeable to both dyes. Viable cells are not stained by either dye. FIG. 3 illustrates the resulting percentages of apoptotic and dead cells. Mean values and standard errors are derived from 3-7 independent experiments.

### Example 11

### Synergistic Effects

AML cells are treated concurrently with SAHA or SNDX-275 and/or bortezomib for 48 hours. IC50 concentrations of HDACIs and 1, 2, 4, or 6 nM or bortezomib are used. Viability of cells is assessed by flow cytometry following staining with YO-PRO®-1-iodide and propidium iodide. FIGS. 4A and 4B illustrate the results.

Molm13 AML cells are treated concurrently with SAHA or SNDX-275 and/or bortezomib for 48 hours. Relative cell number is assessed by MTT assay. Interactions between HDACI and bortezomib therapies are assessed using median effect analysis. Combination indices are shown in FIGS. 5A and 5B.

What is claimed is:

1. A method for treating cancer comprising administering to a patient a therapeutically effective amount of a Class I selective HDAC inhibitor and a proteasome inhibitor.

2. The method of claim 1, wherein the proteasome inhibitor is selected from bortezomib (Velcade, PS-341), PR-171 (carfilzomib), and NPI-0052 (salinosporamide A).

3. The method of claim 1, wherein the proteasome inhibitor is bortezomib.

4. The method of claim 1, wherein the Class I selective HDAC inhibitor is selected from N-(2-amino-phenyl)-4-[(4-pyridin-3-yl-pyrimidin-2-ylamino)-methyl]-benzamide (MGCD-0103), N-(2-aminophenyl)-4-(N-(pyridin-3-ylmethoxycarbonyl)aminomethyl)benzamide (MS-275, SNDX-275), FK228, spiruchostatin A, SK7041, SK7068 and 6-amino nicotinamides.

5. The method of claim 4, wherein the Class I selective HDAC inhibitor is N-(2-aminophenyl)-4-(N-(pyridin-3-ylmethoxycarbonyl)aminomethyl)benzamide.

6. The method of claim 4, wherein the Class I selective HDAC inhibitor is N-(2-amino-phenyl)-4-[(4-pyridin-3-yl-pyrimidin-2-ylamino)-methyl]-benzamide.

7. The method of claim 1, wherein the Class I selective HDAC inhibitor forces G₁ arrest.

8. The method of claim 1, wherein the proteasome inhibitor is administered after the Class I selective HDAC inhibitor.

9. The method of claim 1, wherein the cancer is multiple myeloma, non-small cell lung cancer, acute myeloid leukemia, lymphoblastic lymphoma, follicular lymphoma, non-Hodgkin's lymphoma, mantle cell lymphoma, lung cancer, Hodgkin's lyphoma, head and neck cancer, colorectal cancer, ovarian cancer, leukemia, prostrate cancer, melanoma, bladder cancer, kidney cancer, lung cancer, sarcoma, gastric cancer, pancreatic cancer, liver cancer, gastrointestinal cancer, cervical cancer and breast cancer.

10. The method of claim 1, further comprising administering at least one additional cancer therapy to the patient.

11. The method of claim 10, wherein the additional cancer therapy is selected from surgery or radiation therapy.

12. The method of claim 10, wherein the additional cancer therapy is administration of a second chemotherapeutic agent.

13. The method of claim 12, wherein the chemotherapeutic agent is adriamycin, gemcitabine, mitomycin C, cisplatin, carboplatin, oxaliplatin, fluorouracil, leucovorin, cytarabine, etoposide, capecitabine, temozolomide, doxorubicin, daunomycin, daunorubicin, paclitaxel, docetaxel, cyclophosphamide, ifosfamide, methotrexate, bevacizumab or trastuzumab.

14. The method of claim 1, wherein the HDAC inhibitor sensitizes the cancer cells to the proteasome inhibitor.

15. A method for treating cancer comprising administering to a patient a therapeutically effective amount of N-(2-aminophenyl)-4-(N-(pyridin-3-ylmethoxycarbonyl)aminomethyl)benzamide and bortezamide.

16. The method of claim 15, wherein the N-(2-aminophenyl)-4-(N-(pyridin-3-ylmethoxycarbonyl)aminomethyl)benzamide is administered after the bortezamide.

17. A method for treating cancer comprising administering to a patient a therapeutically effective amount of N-(2-aminophenyl)-4-(N-(pyridin-3-ylmethoxycarbonyl)aminomethyl)benzamide and salinosporamide A.

18. The method of claim 14, wherein the N-(2-aminophenyl)-4-(N-(pyridin-3-ylmethoxycarbonyl)aminomethyl)benzamide is administered after the salinosporamide A.

19. A kit comprising a therapeutically effective amount of a Class I selective HDAC inhibitor and a proteasome inhibitor.

20. The kit of claim 19, wherein the proteasome inhibitor is selected from bortezomib (Velcade, PS-341), PR-171 (carfilzomib) and NPI-0052 (salinosporamide A).

21. The kit of claim 20, wherein the Class I selective HDAC inhibitor is selected from N-(2-amino-phenyl)-4-[(4-pyridin-3-yl-pyrimidin-2-ylamino)-methyl]-benzamide (MGCD-0103), N-(2-aminophenyl)-4-(N-(pyridin-3-ylmethoxycarbonyl)aminomethyl)benzamide (MS-275, SNDX-275), and FK228.

22. The kit of claim 21, wherein the selective HDAC inhibitor is formulated into a first dosage form with a first color and the proteasome inhibitor is formulated into a second dosage form with a second color and wherein the first and second colors are different.

23. The kit of claim 21, comprising at least one dosage form comprising the Class I selective HDAC inhibitor and the proteasome inhibitor.
